(19) United States
(12) Patent Application Publication
(10) Pub. No.: US 2008/0003282 A1
(43) Pub. Date: Jan. 3, 2008

(54) Antiparasitical agents and methods for treating, preventing and controlling external parasites in animals

(76) Inventors: Mark D. Soll, Alpharetta, GA (US); Albert Boeckh, Cumming, GA (US); Krishan Kumar, Manalapan, NJ (US); Natalya Shub, Bridgewater, NJ (US)

Correspondence Address: FROMMER LAWRENCE & HAUG, 745 FIFTH AVENUE- 10TH FL., NEW YORK, NY, 10151, US

(21) Appl. No.: 11/580,731

(22) Filed: Oct. 13, 2006

Related U.S. Application Data
(63) Continuation-in-part of application No. PCT/US05/13097, filed on Apr. 18, 2005.

(51) Int. Cl. A01N 25/34; A01N 43/56; A01P 15/00; C07D 231/02

(52) U.S. Cl. 424/464; 514/407; 548/367.4

(57) ABSTRACT

This invention provides for antiparasitical agents and methods for treating, preventing and controlling external parasites in animals.

### INCORPORATION BY REFERENCE

This application is a continuation-in-part application of international patent application Ser. No. PCT/US2005/013097 filed Apr. 18, 2005, which published as PCT Publication No. WO 2005/099453 on Oct. 27, 2005, which claims priority to U.S. patent application Ser. No. 10/826,105 filed Apr. 16, 2004.

This application also makes reference to U.S. application Ser. No. 10/120,691, filed Apr. 11, 2002, now U.S. Pat. No. 6,797,724, which is a continuation-in-part of U.S. application Ser. No. 08/933,016, filed Sep. 18, 1997, now U.S. Pat. No. 6,010,710, which is in turn a continuation-in-part of application U.S. Ser. No. 08/692,178, filed Aug. 5, 1996, now abandoned, which claims priority to, French patent application No. 96 04 209 filed Mar. 29, 1996, and French patent application No.97 03 708 filed Mar. 26, 1997; and this application is also a continuation-in-part of U.S. application Ser. No. 09/051,693, filed Jul. 27, 1998, now U.S. Pat. No. 6,001,384, which in turn is the National Phase of International Application PCT/FR97/01504 having an international filing date of Aug. 19, 1997, and designating the U.S. and claiming priority from French patent application No. 96 10 312, filed Aug. 20, 1996.

Reference is also made to: U.S. application Ser. No. 09/271,470 filed Mar. 17, 1999, now U.S. Pat. No. 6,482,425, which is a continuation-in-part of International Application PCT/FR97/01548, having an international filing date of Sep. 15, 1997 and designating the U.S., and claiming priority to French application No. 96 11 446 filed Sep. 19, 1996; U.S. application Ser. No. 09/376,736, filed Aug. 17, 1999, U.S. Pat. No. 6,426,333, which is a continuation-in-part of U.S. application Ser. No. 09/271,470 filed on Mar. 17, 1999, now U.S. Pat. No. 6,482,425, which is a continuation-in-part of International Application PCT/FR97/01548, having an international filing date of Sep. 15, 1997 and designating the U.S., and claiming priority to French application No. 96 11 446 filed Sep. 19, 1996; U.S. application Ser. No. 09/381,794, filed Sep. 24, 1999, now U.S. Pat. No. 6,716,442, which in turn is the National Phase of International Application No. PCT/FR98/00601 having an international filing date of Mar. 25, 1998 and designating the U.S., and claiming priority to French application No. 97 03 709, filed Mar. 26, 1997; U.S. application Ser. No. 08/891,047, filed Jul. 10, 1997, now U.S. Pat. No. 6,162,820, which claims priority from French application No. 96 08 703 filed Jul. 11, 1996 and French application No. 97 03 025 filed Mar. 13, 1997; U.S. application Ser. No. 08/863,692 filed Mar. 27, 1997, now U.S. Pat. No. 6,096,329, which is a continuation-in-part of U.S. application Ser. No. 08/692,113 filed Aug. 5, 1996, now abandoned, which claims priority to French patent application 96 04 208, filed Mar. 29, 1996 and French application No. 97 03 711 filed Mar. 26, 1997; U.S. application Ser. No. 08/719,942 filed Sep. 25, 1996, now U.S. Pat. No. 6,395,765, which claims priority to French application No. 95 11 685 filed Sep. 29, 1995 and French application No. 96 11 278 filed Sep. 11, 1996; and U.S. application Ser. No. 09/174,598 filed Oct. 19, 1998, now U.S. Pat. No. 6,083,519, which is a divisional application of U.S. application Ser. No. 08/863,182 filed Mar. 27, 1997, now U.S. Pat. No. 5,885,607, which is a continuation-in-part of U.S. application Ser. No. 08/692,430 filed Aug. 5, 1996, now abandoned, and which claims priority to French application No. 96 04 206 filed Mar. 29, 1996 and French application No. 97 03 707 filed Mar. 26, 1997.

Each of the herein cited patent applications, and all documents cited in the text or during the prosecution of the herein cited patent applications (“application cited documents”) —either cited by the Examiner (e.g., Patent Office, such as the U.S. or French Patent Office or WIPO or Searching Authority) or by the applicant(s)—as well as all documents cited or referenced in application cited documents, are hereby incorporated herein by reference. Further, all documents cited herein (“herein cited documents”) and all documents cited or referenced in herein cited documents are hereby incorporated herein by reference.

Further, the following documents are hereby incorporated herein by reference:
U.S. Pat. No. 5,885,607, issued to Jeannin on Mar. 23, 1999;
U.S. Pat. No. 5,801,189, issued to Twinn on Sep. 1, 1998;
U.S. Pat. No. 5,232,940, issued to Hatton on Aug. 3, 1993;
U.S. Pat. No. 5,122,530, issued to Tornioka on Jun. 16, 1992;
U.S. Pat. No. 5,567,429, issued to Senbo on Oct. 22, 1996;
U.S. Pat. No. 4,963,575, issued to Buntain on Oct. 16, 1990;
U.S. Pat. No. 5,516,787, issued to Takada on May 14, 1996;
U.S. Pat. No. 5,629,334, issued to Takada on May 13, 1997;
EP 0 295 117 with an international filing date of Jun. 10, 1988;
PCT/EP98/01224 with an international filing date of Mar. 1, 1998;
PCT/EP97/06503 with an international filing date of Nov.21, 1997;
Bloomquist, J. R., Ion Channels as Targets for Insecticides, Annu. Rev. Entomol; Vol. 41,163-90 (1996);
Hainzl, D. et al, Mechanisms for Selective Toxicity of Fipronil Insecticide and Its Sulfone Metabolite and Desulfinyl Photoproduct, Chem. Res. Toxicol; Vol. 11(12), 1529-35 (1998);
Hainzi, D. et al, Fipronil Insecticide: Novel Photochemical Desulfinylation with Retention of Neurotoxicity, Proc. Natl. Acad, Sci. USA; Nov. 12;93(23), 12764-7 (1996); and Cochet, P. et al, Skin Distribution of Fipronil by Microautoradiography following Topical Administration to the Beagle Dog, Eur. J. Drug Metab. Pharmacokinet. Jul-Sep; 22(3): 211-6 (1997).

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. Citation or identification of any document in this application is not an admission that such document is available as prior art to the present invention.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. The embodiments of the present invention are disclosed herein or are obvious from and encompassed by, the detailed description. The detailed description, are given by way of example and not intended to limit the invention solely to the specific embodiments described.

### DESCRIPTION OF THE INVENTION

This invention envisions compounds, compositions, formulations and methods of use involving a phenylpyrazole as depicted herein or in herein cited documents; and phenylpyrazoles depicted herein or in herein cited documents can be used in the compositions, formulations and methods of herein cited patent applications.

Further, the invention envisions compounds that degrade, e.g., biodegrade or photodegrade or chemically degrade, to phenylpyrazoles as depicted herein or in herein cited documents or to same or similar derivatives of phenylpyrazoles herein depicted from degradation (e.g., biodegradation, photodegradation, chemical degradation); for instance, if a herein depicted phenylpyrazole degrades to compound “X”, the invention envisions compounds “X” and other phenylpyrazoles that degrade to compound “X”.

The invention also envisions compositions, formulations, and methods of use involving compounds that degrade to phenylpyrazoles as depicted herein or in herein cited documents or to same or similar derivatives of phenylpyrazoles herein depicted from degradation; for example, pour-on formulations, spot-on formulations, formulations and/or methods for distribution in sebaceous glands, formulations and/or methods of preventing, treating, controlling, and/or combating fleas, ticks, lice, mites, flies, mosquitoes, myiasis, and the like, as well as in compositions, formulations, and uses of herein cited patent applications. (A compound that degrades to a phenylpyrazole herein depicted or that degrades to a same or similar derivative of a phenylpyrazole herein depicted from degradation, e.g., a phenylpyrazole that degrades to compound “X” that is a degradation derivative of a herein depicted phenylpyrazole is herein termed a “pro-pp-compound”.)

More in particular, herein depicted phenylpyrazoles as well as those of documents cited herein, e.g., PCT/EP97/06503 or WO 98/24769, which is equivalent to U.S. Pat. No. 6,526,430, degrade to the active compound fipronil sulfide (sulfide on the 4 position) and thus, the invention comprehends the use of phenylpyrazoles depicted herein and in documents cited herein that degrade to fipronil sulfide, in for example, pour-on formulations, spot-on formulations, formulations and/or methods for distribution in sebaceous glands, formulations and/or methods of preventing, treating, controlling, and/or combating fleas, ticks, lice, mites, flies, mosquitoes, myasis, and the like, as well as in compositions, formulations, and uses of herein cited patent applications. Ethiprol (or a 4-ethylsulfoxide derivative of herein depicted phenylpyrazole) as well as compounds of the herein formula wherein there is a sulfide at the 4 position and/or R₁═CN, SO and/or R₃═CF₃ or alkyl (e.g., 4-haloalkyl sulfoxide sulfones, 3-cyanos, 3-cyano-pro-pp-compounds) are useful, e.g., in pour-on formulations and methods.

Formulations, compositions and methods of use can include an additional active ingredient, such as a macrocyclic lactone and/or an insect growth regulator (IGR), or the like, which can be admixed with or administered separately from, e.g., sequentially, with the phenylpyrazole and/or pro-pp-compound (or the macrocyclic lactone and/or IGR can itself, without the phenylpyrazole and/or pro-pp-compound, be the active ingredient in the formulation, such as pour-on or spot-on formulation or a formulation for distribution in the sebaceous glands, as disclosed herein or in herein cited patent applications). These and other embodiments are provided in this text.

The present invention relates to, inter alia, a direct pour-on or spot-on skin solutions or a spray, which contains an antiparasitic product or products and is intended to be applied topically to animals, such as cattle, sheep, and companion animals such as dogs and cats. This invention also relates to oral formulations, such as chewable veterinary formulations or tablets.

The invention also relates to the use of antiparasitic compounds for the preparation of these skin solutions, as well as to a treatment processes relating thereto.

Animals, such as cattle, sheep and companion animals are affected by a large number of parasites, such as fleas, ticks, lice, mites, gnats, flies and mosquitoes.

For cattle and sheep, the main ones are ticks of the genus Boophilus, among which mention may be made of the species microplus (cattle tick), decoloratus and anulatus.

The other main parasites of cattle and sheep are indicated in order of decreasing importance:

myiases such as Dermalobia hominis (known as Berne in Brazil) and Cochlyomia hominivorax (greenbottle); sheep myiases such as Lucilia sericata, Lucilia cuprina (known as blowfly strike in Australia, New Zealand and South Africa). These are flies whose larva constitutes the animal parasite.

flies proper, namely those whose adult constitutes the parasite, such as Haematobia irritans (horn fly).

lice such as Linognathus vitulorum, etc.

mites such as Sarcoptes scabiei and Psoroptes ovis.

mosquitoes such as Aedes sp. and Culex sp.

For companion animals, such as dogs and cats

Ticks: Rhipicephalus sanguineus, Dermacentor variabilis, D. reticulatus, Amblyomma americanum, A. hebreum, A. cajennense, Ixodes scapularis, I. ricinus, I. dammini Haemaphysalis longicornis, H. flava, H. leachi and others

Fleas: Ctenocephalides felis, C. Canis and others

Lice: Felicola subrostratus, Trichodectes canis and others

Mange: Sarcoptes scabei and others

Ticks, in particular Boophilus microplus, are very closely attached to the pasture in which they live and are particularly difficult to control. Similarly, Rhipicephalus sp is closely attached to kennel environments and also difficult to control.

WO-A-87/3781, EP-A-295,117 and EP-A-500,209 describe a class of insecticides which are N-phenyl-pyrazole derivatives. These compounds are given as having activity against a very large number of parasites, including Boophilus microplus, in fields as varied as agriculture, public health and veterinary medicine. The general teaching of these documents indicates that these insecticidal compounds may be administered via different routes; oral including, baits, tablets, chewables and dietary supplements, parenteral; percutaneous and topical routes. Topical administration comprises, in particular, skin solutions (pour-on), solutions for spraying (sprays), baths, showers, jets, powders, greases, shampoos, creams, etc. The pour-on type skin solutions are designed for percutaneous administration. Example 9 of EP-A-295,117 and Example 29I of EP-A-500,209 describe a pour-on type skin solution containing 15% insecticide and 85% dimethyl sulphoxide, for percutaneous administration of the insecticide.

EP-A-296,381 also describes pyrazole compounds having insecticidal activity in the field of agriculture, public health and veterinary medicine. Boophilus microplus is one of the very many targets mentioned. There are very many forms of administration here also, and these include, for example, solutions, emulsions, suspensions, powders, pastes, granules and aerosols.

Other methods for administering pyrazole compounds include placing the therapeutic agent in a solid or liquid matrix for oral delivery. These methods include chewable drug-delivery formulations. The problem associated with oral formulations is that the therapeutic agent often provides an unpleasant taste, aroma, or mouth feel to the formulation, which cause, especially in the situation with animals, the oral formulation to be rejected by the patient. See, e.g., U.S. Pat. No. 5,380,535 to Geyer et al., which provides for a lipid based, chewable formulations for oral delivery of therapeutic agents, such as aspirin, ibuprofen or erythromycin, which are unpalatable to humans; U.S. Pat. No. 5,894,029 to Brown et al., which provides for dried puff pet foods comprising farinaceious materials, proteinaceous materials, such as meats or vegetable protein sources, and optionally medicaments or vitamins; or U.S. Pat. No. 5,637,313 to Chau et al., which describes chewable dosage forms comprising a water soluble matrix comprising hydrogenated starch hydrolystate bulking agent and a water insoluble bulking agent.

Traditionally, in oral veterinary formulations, palatability had been achieved by the inclusion of animal byproducts or flavors derived from animal sources into the formulation. For example, it is customary to include attracts, such as chicken powder, liver powder, beef, ham, fish, or rawhide-derived products in dog chews to make the chew palatable to the dog. See, e.g., U.S. Pat. No. 6,086,940;.U.S. Pat. No. 6,093,441; U.S. Pat. No. 6,159,516; U.S. Pat. No. 6,110,521; U.S. Pat. No. 5,827,565; U.S. Pat. No. 6,093,427, all to Axelrod et al. However, the use of animal products or byproducts or flavors derived from animal sources have recently fallen into disfavor because of the possibility of chemical or biological contamination, which lead to toxicity or diseases such as bovine spongiform encephalopathy. Hence, there is a need for oral veterinary formulations that do not contain animal products, byproducts, or flavors derived from animal sources while still exhibiting good organoleptic properties. While non-animal derived products such as valerian plants are know as scent attractants in food products or pet toys (U.S. Pat. No. 5,785,382 to Childers-Zadah) or animal chews that contain fruit flavors as the attractant (see, U.S. Pat. Nos. 6,274,182; 6,200,616 and 6,126,978 to Axelrod et al.), these patents do not describe using valerian plants or fruit flavors in oral formulations in which the pharmaceutical agents needs to be masked.

An objective of the present invention is to find an effective means which is entirely suitable for controlling parasites of animals, especially companion animals, cattle and sheep, in particular fleas, ticks, lice, gnats, flies and mosquitoes most particularly Boophilus microplus in cattle and in particular lice and blowfly in sheep, Rhipicephalus sp. and Ctenocephalides sp. in dogs and cats under the conditions in which these animals are reared.

Applicants have found that it is possible to effectively control fleas and ticks in companion animals and Boophilus microplus for cattle using a topical or oral formulation according to the present invention. Applicants have also found that this formulation is effective against sheep lice and sheep flies known as “blowfly.” An aim of the present invention is thus to provide a novel composition and method which is entirely effective against fleas and ticks in animals, including Boophilus microplus and also against all of the other parasites described above such as, in particular, sheep lice and “blowfly”, these compositions are entirely suitable for controlling these parasites under the conditions in which these animals are reared.

Another aim of the invention is to provide such a formulation, which has a long period of efficacy, preferably longer than or equal to one or two months.

Another aim of the invention is to provide such a formulation; which is quick and easy to use and entirely compatible with use on herds or flocks containing a large number of animals.

Another aim of the invention is to provide such a formulation, which is particularly suitable for extensive pasture rearing and for use intended to protect animals during the period of rounding up and finishing (Feed Lot in USA), namely the final period of rearing in which a large number of animals are herded into a small enclosure over an average period of two months preceding slaughter.

This invention further provides for a process for preparing 1-N-arylpyrazole compounds, which also possess activity against parasites, such as fleas and ticks, in animals, such as cats, dogs, zebras, llamas, horses, cattle and sheep. These compounds include compounds of the formula

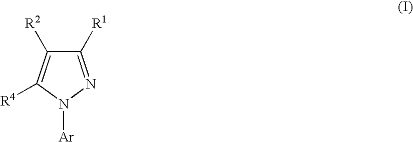

in which:
R¹ is CN;
R² is S(O)ₙR³ or 4,5-dicyanoimidazol-2-yl or haloalkyl;
R³ is alkyl, haloalkyl, haloalkenyl or halogenalkenyl;
R⁴ represents a hydrogen, alkyl, haloalkyl, amino, or a compound selected from the group consisting of

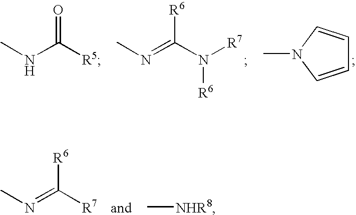

wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl, or a radical NR⁹R⁹R¹⁰, S(O)ₘR¹¹, C(O)R¹¹, C(O)O—R^{11,} alkyl, haloalkyl, or
OR₁₂, or a radical —N═C(R¹³) (R¹⁴); wherein
R⁹ and R¹⁰ independently represent a hydrogen atom or an alkyl, haloalkyl, C(O)alkyl, alkoxycarbonyl or S(O)ᵣCF₃ radical; or R⁹ and R¹⁰ may together form a divalent alkylene radical which may be interrupted by one or two divalent hetero atoms, such as oxygen or sulphur;
R¹¹ represents an alkyl or haloalkyl radical;
R¹² represents an alkyl or haloalkyl radical or a hydrogen atom;
R¹³ represents an alkyl radical or a hydrogen atom;
R¹⁴ represents a phenyl or heteroaryl group optionally substituted with one or more halogen atoms or groups such as OH,—O-alkyl, —S-alkyl, cyano or alkyl;
Ar represents unsubstituted or substituted phenol or pyridyl and
m, n, and r represent, independently of each other, an integer equal to 0, 1 or 2.

In another embodiment of the invention, the compounds include compounds of the formula

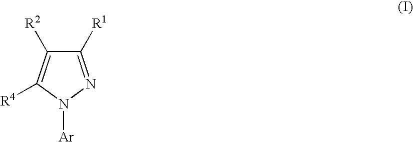

in which:
R¹ is CN;
R² is S(O)ₙR³ or 4,5-dicyanoimidazol-2-yl or C₁-C₆ haloalkyl;
R³ is C₁-C₆ alkyl, C₁-C₆ haloalkyl, C₂-C₆ haloalkenyl or C₂-C₆ halogenalkenyl;
R⁴ represents a hydrogen, C₁-C₆ alkyl, C₁-C₆ haloalkyl, amino, or a compound selected from the group consisting of 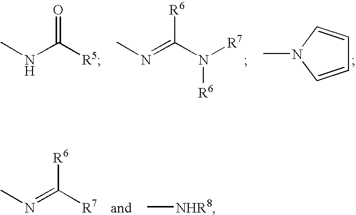
wherein
   R⁵ represents C₁-C₆ alkyl, C₁-C₆ halogenoalkyl, C₁-C₆ alkoxyalkyl or in each case unsubstituted or substituted phenyl or pyridyl, wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano;
   R⁶ represents hydrogen or C₁-C₆ alkyl,
   R⁷ represents hydrogen, C₁-C₆ alkyl or in each case unsubstituted or substituted phenyl or pyridyl, wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano;
   R⁸ represents C₁-C₆ alkyl, C₂-C₆ alkenyl, C₂-C₆ alkynyl, formyl, C₁-C₆ alkylcarbonyl, C₁-C₆ halogenoalkylcarbonyl, or C₁-C₆ alkoxycarbonyl, or a radical NR⁹R⁹R¹⁰, S(O)ₘR¹¹, C(O)R¹¹, C(O)O—R¹¹, alkyl, haloalkyl, or
   OR₁₂, or a radical —N═C(R¹³) (R¹⁴) ; wherein
   R⁹ and R¹⁰ independently represent a hydrogen atom or an C₁-C₆ alkyl, C₁-C₆ haloalkyl, C(O) C₁-C₆ alkyl, alkoxycarbonyl or S(O)ᵣCF₃ radical; or
   R⁹ and R¹⁰ may together form a divalent C₁-C⁶ alkylene radical which may be interrupted by one or two divalent hetero atoms, such as oxygen
   or sulphur;
   R¹¹ represents an C₁-C₆ alkyl or C₁-C₆ haloalkyl radical;
   R¹² represents an C₁-C₆ alkyl or C₁-C₆ haloalkyl radical or a hydrogen atom;
   R¹³ represents an C₁-C₆ alkyl radical or a hydrogen atom;
   R¹⁴ represents a phenyl or heteroaryl group optionally substituted with one or more halogen atoms or groups such as OH,—O—C₁-C₆ alky, —S—C₁-C₆ alkyl, cyano or C₁-C₆ alkyl;
Ar represents unsubstituted or substituted phenol or pyridyl wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano; and
m, n, and r represent, independently of each other, an integer equal to 0, 1 or 2.

A preferred value for Ar is 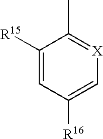
where
R¹⁵ and R¹⁷ represent, independently of each other, a hydrogen or halogen atom, or optionally CN or NO₂;
R¹⁶ represents a halogen atom or a haloalkyl, haloalkoxy, S(O)_{q}CF₃ or SF₅ group;
q represents an integer equal to 0, 1 or 2;
X represents a trivalent nitrogen atom or a radical C—R¹⁷, the other three valency positions of the carbon atom forming part of the aromatic ring.

Another embodiment for Ar is 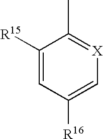
where
R¹⁵ and R¹⁷ represent, independently of each other, a hydrogen or halogen atom, or optionally CN or NO_{2;}
R¹⁶ represents a halogen atom or a C₁-C₆ haloalkyl, C₁-C₆ haloalkoxy,
S(O)_{q}CF₃ or SF₅ group;
q represents an integer equal to 0, 1 or 2;
X represents a trivalent nitrogen atom or a radical C—R¹⁷, the other three valency positions of the carbon atom forming part of the aromatic ring;

The compound of formula (I) which are most preferably in a formulation at low volume (preferably from 0.05 to 25% weight volume) in pour-on and spot-on formulas that are designed to release the compound (II) onto the skin and the hairs for a contact action against parasites.

Preferably, in formula (I),
R¹ is CN;
R² is S(O)ₙR₃;
R³ is alkyl or haloalkyl;
R⁴ represents a hydrogen or halogen atom; or a radical NR⁹R¹⁰, S(O)ₘR¹¹, C(O)R¹¹, alkyl, haloalkyl or OR¹² or a radical —N═C (R¹³) (R¹⁴);
R⁹ and R¹⁰ independently represent a hydrogen atom or an alkyl, haloalkyl, C(O)alkyl or S(O)ᵣCF₃ radical; or R⁹ and R¹⁰ may together form a divalent alkylene radical which may be interrupted by one or two divalent hetero atoms, such as oxygen or sulphur;
R¹¹ represents an alkyl or haloalkyl radical;
R¹² represents an alkyl or haloalkyl radical or a hydrogen atom;
R¹³ represents an alkyl radical or a hydrogen atom;
R¹⁴ represents a phenyl or heteroaryl group optionally substituted with one or more halogen atoms or groups such as OH, —O-alkyl, —S-alkyl, cyano or alkyl;
R¹⁵ and R¹⁷ represent, independently of each other, a hydrogen or halogen atom;
R¹⁶ represents a halogen atom or a haloalkyl, haloalkoxy, S(O)_{q}CF₃ or SF₅ group;
m, n, q, and r, represent, independently of each other, an integer equal to 0, 1 or2;
X represents a trivalent nitrogen atom or a radical C—R¹⁷ the other three valency positions of the carbon atom forming part of the aromatic ring.

More preferably, in formula (I),
R¹ is CN;
R² is S(O)ₙR₃;
R³ is alkyl or C₁-C₆ haloalkyl;
R⁴ represents a hydrogen or halogen atom; or a radical NR⁹R¹⁰, S(O)ₘR¹¹, C(O)R¹¹, C₁-C₆ alkyl, C₁-C₆ haloalkyl or OR¹² or a radical —N═C (R¹³) (R¹⁴);
R⁹ and R¹⁰ independently represent a hydrogen atom or an C₁-C₆ alkyl, C₁-C₆ haloalkyl, C(O)—C₁-C₆ alkyl or S(O)ᵣCF₃ radical; or R⁹ and R¹⁰ may together form
a divalent alkylene radical which may be interrupted by one or two divalent hetero atoms, such as oxygen or sulphur;
R¹¹ represents an C₁-C₆ alkyl or C₁-C₆ haloalkyl radical;
R¹² represents an C₁-C₆ alkyl or C₁-C₆ haloalkyl radical or a hydrogen atom;
R¹³ represents an c₁-C₆ alkyl radical or a hydrogen atom;
R¹⁴ represents a phenyl or heteroaryl group optionally substituted with one or more halogen atoms or groups such as OH, —O—C₁-C₆-alkyl, —S—C₁-C₆-alkyl, cyano or C₁-C₆ alkyl;
R¹⁵ and R¹⁷ represent, independently of each other, a hydrogen or halogen atom;
R¹⁶ represents a halogen atom or a C₁-C₆ haloalkyl, C₁-C₆ haloalkoxy, S(O)_{q}CF₃
or SF₅ group;
m, n, q, and r represent, independently of each other, an integer equal to 0, 1 or 2;
X represents a trivalent nitrogen atom or a radical C—R¹⁷ the other three valency positions of the carbon atom forming part of the aromatic ring.

This invention provides for a process for the control or elimination of external parasites from an animal, comprising topically applying or orally administering a parasitically effective amount of a formulation comprising a compound of the formula:

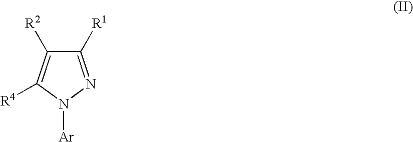

wherein:

R¹ represents H₂N—CS—;

R² represents S(O)ₙR, 4,5-dicyanoimidazol-2-yl or haloalkyl;

R³ represents alkyl, haloalkyl, haloalkenyl or haloalkynyl;

R⁴ represents hydrogen, halogen, alkyl, haloalkyl, amino or a compound selected from the group consisting of 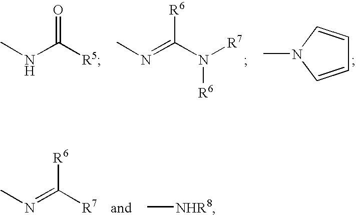
wherein
   R⁵ represents alkyl, halogenoalkyl, alkoxyalkyl or in each case unsubstituted or substituted phenyl or pyridyl,
   R⁶ represents hydrogen or alkyl,
   R⁷ represents hydrogen, alkyl or in each case unsubstituted or substituted phenyl or pyridyl,
   R⁸ represents alkyl, alkenyl, alkynyl, formyl, alkylcarbonyl,
   halogenoalkylcarbonyl, or alkoxycarbonyl,
   or a radical NR⁹R⁹R¹⁰, S(O)ₘR¹¹, C(O)R¹¹, C(O)R¹¹, OR¹², or —N═C(R¹³)(R¹⁴)
   wherein
   R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
   R¹¹ represent an alkyl or haloalkyl radical;
   R¹² represents an alkyl or haloalkyl radical or a hydrogen atom;
   R¹³ represents an alkyl radical or a hydrogen atom;
   R¹⁴ represents a phenyl or a heteroaryl group optionally substituted with one or more halogen atoms or OH, —O-alkyl, S-alkyl, cyano or alkyl; m, n q, represents independently of each other an integer equal to 0, 1, or 2;
Ar represents unsubstituted or substituted phenyl or pyridyl, and
n represents a number 0, 1 or 2; and subjecting the formulation to degradation while diffusing therefrom over the animal's body and/or in the sebaceous glands of the animal and thereby obtaining said control or elimination of said external parasites.

This invention also provides for, a method for the control or elimination of external parasites from an animal, comprising topically applying, at least monthly, to a localized region on the back of the animal, a parasitically effective amount of a direct pour-on or spot-on formulation comprising from 0.05 to 25% weight/volume, relative to the total solution, of a compound of the formula:

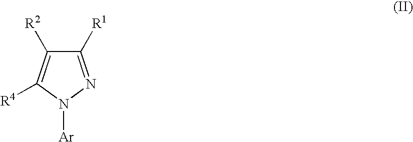

wherein:

R¹ represents H₂N—CS—;

R² represents S(O)ₙR³, 4,5-dicyanoimidazol-2-yl or haloalkyl;

R³ represents alkyl, haloalkyl, haloalkenyl or halogenalkynyl;

R⁴ represents hydrogen, halogen, alkyl, amino or a compound selected from the group consisting of 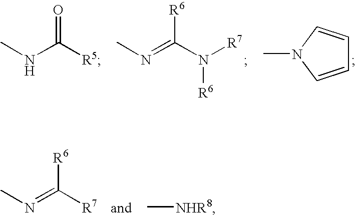
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl,

or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹¹ represents an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and
n represents a number 0, 1 or 2,
   wherein the preferably composition comprises from 0.05 to 25% weight/volume of the compound of formula (II), and the compound of formula (II) is applied in a dose between 0.1 and 2 mg/kg animal weight, and subjecting the formulation to degradation while diffusing therefrom over the animal's body and/or in the sebaceous glands of the animal and subjecting thereby obtain said control or elimination of said external parasites.

Preferably, the parasitically effective amount of a direct pour-on or spot-on formulation comprises a compound of the formula:

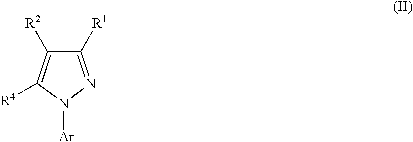

wherein:

R¹ represents H₂N—CS—;

R² represents S(O)ₙR³, 4,5-dicyanoimidazol-2-yl or C₁-C₆ haloalkyl;

R³ represents C₁-C₆ alkyl, C₁-C₆ haloalkyl, C₂-C₆ haloalkenyl or C₂-C₆ halogenalkynyl;

R⁴ represents hydrogen, halogen, C₁-C₆ alkyl, amino or a compound selected from the group consisting of 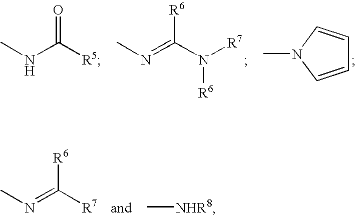
wherein
R⁵ represents C₁-C₆ alkyl, C₁-C₆ halogenoalkyl, C₁-C₆-alkoxy-C₁-C₆-alkyl or in each case unsubstituted or substituted phenyl or pyridyl, wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano;
R⁶ represents hydrogen or C₁-C₆ alkyl,
R⁷ represents hydrogen, alkyl or in each case unsubstituted or substituted phenyl or pyridyl, wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano;
R⁸ represents C₁-C₆ alkyl, C₁-C₆ alkenyl, C₂-C6 alkynyl, formyl, C₁-C₆ alkylcarbonyl, C₁-C₆ halogenoalkylcarbonyl, or C₁-C₆ alkoxycarbonyl, or a radical NR⁹R⁹R¹⁰, S(O)R¹¹, C(O)R¹¹, C(O)R¹¹, OR¹², or —N═C(R¹³)(R¹⁴)
wherein
R⁹ and R¹⁰ independently represent a hydrogen atom or an C₁-C₆ alkyl, C₁-C₆ haloalkyl, C(O)—C₁-C₆-alkyl, C₁-C₆ alkyoxycarbonyl or a S(O)ᵣCF₃ radical; or
R⁹ and R¹⁰ may together form a divalent alkenyl radical which may be interrupted
by one or two heteroatoms;
R¹¹ represents an C₁-C₆ alkyl or C₁-C₆ haloalkyl radical;
R¹² represents an C₁-C₆ alkyl or C₁-C₆ haloalkyl radical or a hydrogen atom;
R¹³ represents an C₁-C₆ alkyl radical or a hydrogen atom;
R¹⁴ represents a phenyl or a heteroaryl group optionally substituted with one or more halogen atoms or OH, —O— C₁-C₆ alkyl, S— C₁-C₆ alkyl, cyano or C₁-C₆ alkyl;
Ar represents unsubstituted or substituted phenyl or pyridyl, wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano; and
m, n, q, r represents independently of each other an integer equal to 0, 1, or 2.

The present invention further provides for improved oral veterinary formulations, which do not contain animal products or flavors derived from animal sources, that exhibit organoleptic properties that the animal finds appealing. This invention further provides for improved chewable veterinary formulations or which do not contain animal products or flavors derived from animal sources and possess good consistency and acceptability by the animal, as well as an improved process to prepare chewable veterinary formulations. This invention further contemplates a premix which a premix formulation comprising at least one compound of formula (II) is mixed with the animal's feed.

Preferably, the premix for an animal feed which comprises:

a) a parasitically effective amount of at least one compound of formula (II);

b) a pharmaceutically acceptable excipient comprising:
i) a pharmaceutically acceptable surfactant;
ii) a pharmaceutically acceptable wax;
iii) a pharmaceutically acceptable antioxidant;
iv) a pharmaceutically acceptable carrier vehicle wherein said vehicle is selected from the group consisting of fine corn cobs, corn meal, citrus meal, fermented residues, ground oyster shells, wheat shorts, molasses solubles, bean mill feed, soy grits, crushed limestone and dried grains;

A more preferred embodiment of a premix according to the present invention is one where:

the pharmaceutically acceptable excipient comprises:
i) about 5 to about 15% (w/w) of a surfactant wherein said surfactant is selected from the group consisting of polyoxyl 40 hydrogenated castor oil, PEG-50 castor oil, PET-60 corn glyceride, PEG-60 almond oil, PEG-40 palm kernel oil, and PEG-60 corn oil;
ii) about 5 to about 25% (w/w) of a wax wherein said wax is selected from the group consisting of distilled monoglycerides, glyceryl tribehenate, glyceryl trimyristate, and hydrogenated coco-glycerides;
iii) about 0.1 to about 2% (w/w) of an antioxidant wherein said antioxidant is selected from the group consisting of butylated hydroxyanisole, butylated hydroxytoluene, ascorbic acid, sodium metabisulphite, propyl gallate, sodium sulfite, sodium thiosulphate, and a mixture thereof;
iv) about 60 to about 80% (w/w) of a pharmaceutically acceptable carrier vehicle wherein said carrier vehicle is selected from the group consisting of fine group corn cobs, crushed limestone, and dried grains.
   In these formulations, the compound of formula (II) is from about 0.01 to about 20% (w/w).

A more preferred premix is one where:

the pharmaceutically acceptable exipient comprises:
i) about 5 to about 15% (w/w) of a surfactant wherein said surfactant is selected from the group consisting of polyoxyl 40 hydrogenated castor oil, PEG-50 castor oil, PEG-60 corn glyceride, PEG-60 almond oil, PEG40 palm kernel oil, and PEG-60 corn oil;
ii) about 5 to about 25% (w/w) of a wax wherein said wax is selected from the group consisting of distilled monoglycerides, glyceryl tribehenate, glyceryl trimyristate, and hydrogenated coco-glycerides;
iii) about 0.1 to about 2% (w/w) of an antioxidant wherein said antioxidant is selected from the group consisting of butylated hydroxyanisole, butylated hydroxytoluene, ascorbic acid, sodium metabisulphite, propyl gallate, sodium sulfite, sodium thiosulphate, and a mixture thereof;
iv) about 60 to about 80% (w/w) of a pharmaceutically acceptable carrier vehicle wherein said carrier vehicle is selected from the group consisting of fine ground corn cobs, crushed limestone, and dried grains.

Alternatively, the premix comprises an effective amount of at least one compound of the formula 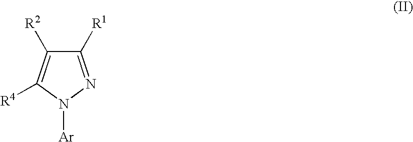
wherein:
R¹ represents H₂N—CS—;
R² represents S(O)ₙR, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of

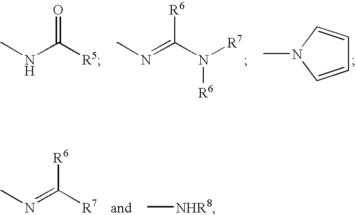

wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl,
or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;

m, n q, r represents independently of each other an integer equal to 0, 1, or 2;

Ar represents unsubstituted or substituted phenyl or pyridyl, and

n represents a number 0, 1 or 2,

—a pharmaceutically acceptable excipient comprising:
i) a pharmaceutically acceptable wax;
ii) a pharmaceutically acceptable antioxidant;
iii) a pharmaceutically acceptable carrier vehicle wherein said vehicle is selected from the group consisting of fine corn cobs, corn meal, citrus meal, fermented residues, ground oyster shells, wheat shorts, molasses solubles, bean mill feed, soy grits, crushed limestone and dried grains;

—an organic solvent and

—optionally a pharmaceutically acceptable pH modifier.

In another alternative embodiment, the premix comprises an effective amount of at least one compound of the formula 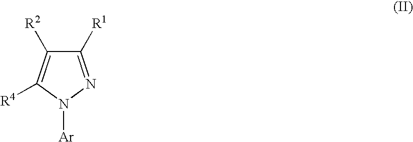
wherein:
R¹ represents H₂N&CS
R² represents S(O)ₙR, 4,5-dicyanoimidazol-2-yl or C₁-C₆ haloalkyl;
R³ represents C₁-C₆ alkyl, C₁-C₆ haloalkyl, C₂-C₆ haloalkenyl or C₂-C₆ haloalkynyl;
R⁴ represents hydrogen, halogen, C₁-C₆ alkyl, C₁-C₆ haloalkyl, amino or a compound selected from the group consisting of

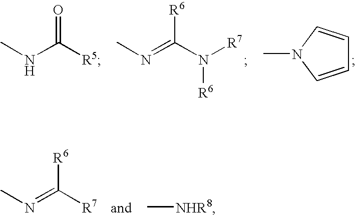

wherein
R⁵ represents C₁-C₆ alkyl, C₁-C₆ halogenoalkyl, C₁-C₆ alkoxy-C₁-C₆ alkyl or in each case unsubstituted or substituted phenyl or pyridyl,
R⁶ represents hydrogen or C₁-C₆ alkyl,
R⁷ represents hydrogen, C₁-C₆ alkyl or in each case unsubstituted or substituted phenyl or pyridyl, wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano;
R⁸ represents C₁-C₆ alkyl, C₂-C₆ alkenyl, a C₂-C₆ alkynyl, formyl, C₁-C₆ alkylcarbonyl, C₁-C₆ halogenoalkylcarbonyl, or C₁-C₆ alkoxycarbonyl,
or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an C₁-C₆ alkyl, C₁-C₆ haloalkyl, C(O) C₁-C₆ alkyl, C₁-C₆ alkoxycarbonyl or a S(O)ᵣCF₃ radical; or
R⁹ and R¹⁰ may together form a divalent alkenyl radical which may be interrupted by one or two heteroatoms;
R¹¹ represent an C₁-C₆ alkyl or C₁-C₆ haloalkyl radical;
R¹² represents an C₁-C₆ alkyl or C₁-C₆ haloalkyl radical or a hydrogen atom;
R¹³ represents an C₁-C₆ alkyl radical or a hydrogen atom;
R¹⁴ represents a phenyl or a heteroaryl group optionally substituted with one or more halogen atoms or OH, —O— C₁-C₆ alkyl, S— C₁-C₆ alkyl, cyano or C₁-C₆ alkyl;

Ar represents unsubstituted or substituted phenyl or pyridyl, wherein the substituents are independently C₁-C₆ alkyl, halogen, C₁-C₆ haloalkyl, C₁-C₆ alkoxy, C₁-C₆ acyl, amino, C₁-C₆ alkylamino, C₁-C₆ dialkylamino, nitro or cyano;

and

m, n, q, r represents independently of each other an integer equal to 0, 1, or 2;

—a pharmaceutically acceptable excipient comprising:
i) a pharmaceutically acceptable wax;
ii) a pharmaceutically acceptable antioxidant;
iii) a pharmaceutically acceptable carrier vehicle wherein said vehicle is selected from the group consisting of fine corn cobs, corn meal, citrus meal, fermented residues, ground oyster shells, wheat shorts, molasses solubles, bean mill feed, soy grits, crushed limestone and dried grains;

—an organic solvent and

—optionally a pharmaceutically acceptable pH modifier.

The organic solvent may be in the form of a liquid carrier vehicle which comprises a solvent and a cosolvent wherein the solvent is selected from the group consisting of acetone, acetonitrile, benzyl alcohol, butyl diglycol, dimethylacetamide, dimethylformamide, dipropylene glycol n-butyl ether, ethanol, isopropanol, methanol, ethylene glycol monoethyl ether, ethylene glycol monomethyl ether, monomethylacetamide, dipropylene glycol monomethyl ether, liquid polyoxyethylene glycols, propylene glycol, 2-pyrrolidone, in particular N-methylpyrrolidone, diethylene glycol monoethyl ether, ethylene glycol, diethyl phthalate fatty acid esters, such as, but not limited to, the diethyl ester or diisobutyl adipate, and a mixture of at least two of these solvents and the cosolvent is selected from the group consisting of absolute ethanol, isopropanol or methanol.

Preferred solvents include diethylene glycol monobutyl ether, propylene glycol, diethylene glycol monoethyl ether, diethylene monobutyl ether and the like.

The antioxidant used in the invention premixes are well known in the art. Examples of antioxidants are but not limited to alpha tocopherol, ascorbic acid, ascrobyl palmitate, fumaric acid, malic acid, sodium ascorbate, sodium metabisulfate, n-propyl gallate, BHA (butylated hydroxy anisole), BHT (butylated hydroxy toluene) monothioglycerol and the like.

The waxes in the inventive premixes are used to protect the active ingredient. Examples of waxes include distilled monoglycerids, glycerol tribehenate, glyceryl trimyristate and hydrogenated coco-glycerides.

A large number of surfactants of different degrees of hydrophobicity or hydrophilicity can be prepared by reaction of alcohols or polyalcohols with a variety of natural and/or hydrogenated oils. Most-commonly, the oils used are castor oil or hydrogenated castor oil, or an edible vegetable oil such as corn oil, olive oil, peanut oil, palm kernel oil, apricot kernel oil, soybean oil, or almond oil. Preferred alcohols include glycerol, propylene glycol, ethylene glycol, polyethylene glycol, sorbitol, and pentaerythritol. Among these alcohol-oil transesterified surfactants, preferred hydrophilic surfactants are PEG-35 castor oil (Incrocas-35), PEG-40 hydrogenated castor oil (Cremophor RH 40), PEG-25 trioleate (TAGAT® TO), PEG-60 corn glycerides (Crovol M70), PEG-60 almond oil (Crovol A70), PEG-40 palm kernel oil (Crovol PK70), PEG-50 castor oil (Emalex C-50), PEG-50 hydrogenated castor oil (Emalex HC-50), PEG-8 caprylic/capric glycerides (Labrasol), and PEG-6 caprylic/capric glycerides (Softigen 767). Preferred hydrophobic surfactants in this class include PEG-5 hydrogenated castor oil, PEG-7 hydrogenated castor oil, PEG-9 hydrogenated castor oil, PEG-6 corn oil (Labrafil® M 2125 CS), PEG-6 almond oil (Labrafil® M 1966 CS), PEG-6 apricot kernel oil (Labrafil® M 1944 CS), PEG-6 olive oil (Labrafil® M 1980 CS), PEG-6 peanut oil (Labrafil® M 1969 CS), PEG-6 hydrogenated palm kernel oil (Labrafil® M 2130 BS), PEG-6 palm kernel oil (Labrafil® M 2130 CS), PEG-6 triolein (Labrafil® M 2735 CS), PEG-8 corn oil (Labrafil® WL 2609 BS), PEG-20 corn glycerides (Crovol M40), and PEG-20 almond glycerides (Crovol A40).

This invention further provides for a method for the control or elimination of external parasites from an animal, comprising topically applying, at least monthly, to an animal a parasitically effective amount of a spray formulation comprising a compound of formula (II).

The formulations contemplated by the present invention, including the premixes and sprays, may further include, among others, an effective amount of at least one other insecticide, ascaricide, insect growth regulator (IGR), nodulisporic acid or a nodulisporic acid derivative, neonicotinoids, such as imidaclopride or nytenpram, pyrethroids such as permethrin, phenothrin, flumethrin, deltamethrinin, cyfluthrin, and others; formamides, such as amitraz and others, and avermectins, such as ivermectin, abamectin, doramectin, emamectin, eprinomectin, latidectin, lepimectin and selamectin, in addition to at least one compound of formula (II)

This invention further provides for a process for preparing 1-N-arylpyrazoles which include compounds of formula I, which comprises subjecting a compound of formula II to a degradation agent such as UV light, fluorescent light, heat or an oxidation agent. This reaction proceeds as follows: 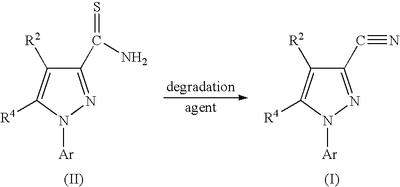
where R², R³ and Ar are defined above.

The expression “pour-on skin solution” or “spot-on skin solution” is understood to refer to a ready-to-use solution intended to be applied topically and locally on the animal, preferably for pour-on formulations on the animal's back and at several points or along the line of the back, and applied in low volume, preferably of about 5 to about 20 ml per 100 kg, preferably about 10 ml per 100 kg, with a total volume of from about 10 to about 150 ml per animal, preferably limited to 50 ml.

1-N-arylpyrazoles act by simple contact, the parasite becoming impregnated with the compound on contact with the hairs and the skin.

This thereby affords, in a noteworthy manner, a both perfect compatibility with the restrictions of use in extensive grazing, in terms of ease of use in particular, and a spectrum of activity and of efficacy, as well as a period of efficacy, which are suited to this type of rearing.

By working on the concentration of the compounds of formula (II), solutions having note-worthy activities are obtained with, in particular, two months of efficacy against Boophilus microplus, this result never before having been achieved. Moreover, the solution according to the invention allows Boophilus microplus to be totally eliminated from an infested animal in less than 2 days. Long lasting efficacy against Ctenocephalides felis, Rhipicephalussonguineus and other species of ticks of economic importance is also achieved.

As has been stated above, the solutions according to the present invention are applied topically, in low volume, to the animal's back. The compounds of formula (II) then convert to fipronil-type compounds, i.e., 1-N-arylpyrazoles which possess a CN moiety on the 3-position of the pyrazole ring, which then diffuse out in a noteworthy manner, this being reflected by a distribution of the compound over the animal's entire body. It has also been observed that the animals remained protected in the case of passage through water or exposure to rain.

The dose of fipronil-type compounds, e.g. compounds of formula (I), is preferably between about 0.1 and about 2 mg/kg (animal weight), preferably between about 0.25 and about 1.5 mg/kg, and in particular about 1 mg/kg. From these guidelines, it is easy to calculate the amount of compounds of formula II that are required to have an effective concentration.

The compounds of formula (I) in which R² is S(O)ₙR³, preferably with n=1, R³ preferably being CF₃ or alkyl, for example methyl or ethyl, or alternatively n=0, R₃ preferably being CF₃, as well as those in which X═C—R¹⁷, R¹⁷ being a halogen atom, will also be selected. The compounds in which R¹⁵ is a halogen atom and those in which R¹⁶ is haloalkyl, preferably CF₃, are also preferred. In the context of the present invention, compounds combining two or more of these characteristics will advantageously be selected.

A preferred class of compounds of formula (I) consists of compounds such that R⁴ is haloalkyl, preferably CF₃, or ethyl, R⁴ is NH₂, R¹⁵ and R¹⁷ are, independently of each other, a halogen atom, and/or R¹⁶ is haloalkyl.

In the present invention, the alkyl radicals may contain generally from 1 to 6 carbon atoms. The cycle formed between the divalent alkylene radical representing R₅ and R₆, as well as with the nitrogen atom to which R₅ and R₆ are attached, may be generally a cycle of 5, 6 or 7 links.

A most particularly preferred compound of the formula (I) in the invention is 1-[2,6-Cl₂ 4-CF₃phenyl]3-CN 4-[SO—CF₃]5-NH₂pyrazole, or fipronil. This compound will be used in particular in a proportion of from about 0.1 to about 2% by weight, more particularly about 1%, relative to the total solution.

Mention may also be made of the two compounds which differ from the above by the following characteristics:

1-n=0, R₃=CF₃

2-n=1, R₃=ethyl.

The compounds of formula (II) may be prepared according to one or other of the processes described in U.S. Pat. No. 6,265,430, or any other process which falls within the competence of a specialist skilled in the art of chemical synthesis. For the chemical preparation of the products of the invention, a person skilled in the art is considered as having at his or her disposal, inter alia, all the contents of “Chemical Abstracts” and the documents cited therein.

It is not departing from the scope of the present invention to incorporate other insecticides into the solution according to the present invention.

Administration of the inventive formulation may be intermittent in time and may be administered daily, weekly, biweekly, monthly, bimonthly, quarterly, or even for longer durations of time. The time period between treatments depends upon factors such as the parasite(s) being treated, the degree of infestation, the type of animal, mammal or bird, and the environment where it resides. It is well within the skill level of the practitioner to determine a specific administration period for a particular situation. This invention contemplates a method for permanently combating a parasite in an environment in which the animal is subjected to strong parasitic pressure where the administration is at a frequency far below a daily administration in this case. For example, it is preferable for the treatment according to the invention to be carried out monthly on mammals, such as on dogs and on cats.

Spot-on formulations may be prepared by dissolving the active ingredients into the pharmaceutically or veterinary acceptable vehicle. Alternatively, the spot-on formulation can be prepared by encapsulation of the active ingredient to leave a residue of the therapeutic agent on the surface of the animal. These formulations will vary with regard to the weight of the therapeutic agent in the combination depending on the species of host animal to be treated, the severity and type of infection and the body weight of the host. The compounds may be administered continuously, particularly for prophylaxis, by known methods. Generally, a dose of from about 0.001 to about 100 mg per kg of body weight given as a single dose or in divided doses for a period of from 1 to 5 days will be satisfactory but, of course, there can be instance where higher or lower dosage ranges are indicated and such are within the scope of this invention. It is well within the routine skill of the practitioner to determine a particular dosing regimen for a specific host and parasite.

Preferably, a single formulation containing the thioamide derivative of the 1-N-aryl pyrazole, e.g. compounds of formula (II), in a substantially liquid carrier and in a form which makes possible a single application, or an application repeated a small number of times, will be administered to the animal over a highly localized region of the animal, preferably between the two shoulders. Remarkably, it has been discovered that such a formulation is highly effective against both the targeted ectoparasites and the targeted endoparasites.

The treatment is preferably carried out so as to administer to the host, on a single occasion, a dose containing between about 0.001 and about 100 mg/kg of a compound of formula (II), with about 10 mg/kg being especially preferred.

The amount of compounds of formula (II) for animals which are small in size is preferably greater than about 0.01 mg and in a particularly preferred way between about 1 and about 50 mg/kg of weight of animal.

It also may be preferable to use controlled-release formulations.

This invention also provides for a method for cleaning the coats and the skin of animals by removal of the parasites which are present and of their waste and excreta. The animals treated thus exhibit a coat which is more pleasing to the eye and more pleasant to the touch.

While not wishing to be bound by theory, it is believed that the invention spot-on formulation work by the compounds of formula (II) at least partly degrading on the animals' skin to 1-N-arylpyrazoles, such as those compounds of formula (II), and the combination of these compounds form an effective dose, which dissolves in the natural oils of the host's skin, fur or feathers. From there, the therapeutic agent(s) distribute around the host's body through the sebaceous glands of the skin. The therapeutic agent also remains in the sebaceous glands. Thus, the glands provide a natural reservoir for the therapeutic agent which allows for the agent to be drained back out to the follicles to reapply itself to the skin and hair. This, in turn, provides for longer time periods between application as well as not having to re-administer the dose after the host becomes wet because of rain, bathes, etc. Moreover, the inventive formulation have the further advantage in self-grooming animals of not being directly deposited of the skin or fur where the animals could orally ingest the therapeutic agent, thereby becoming sick or possibly interacting with other therapeutic agent being orally administered.

The invention also relates to such a method with a therapeutic aim intended for the treatment and prevention of parasitoses having pathogenic consequences.

In another preferred embodiment this provides for a composition for combating fleas in mammals, in particular dogs and cats, characterized in that it contains at least one of formula (II) as defined above.

The effective amount in a dose is, for the compounds of formula (II), preferably between about 0.001, preferentially about 0.1, and about 100 mg/kg and in a particularly preferred way from about 1 to about 50 mg/kg of weight of animal, the higher amounts being provided for very prolonged release in or on the body of the animal.

The formulations of the present invention provide for the topical administration of a concentrated solution, suspension, microemulsion or emulsion for intermittent application to a spot on the animal, generally between the two shoulders (solution of spot-on type). It has been discovered that the inventive formulations are especially active against parasites when the formulations are applied to animals, such as mammals, especially dogs, cats, sheep, pigs, cattle and horses. The thioamides can advantageously be present in this formulation in a proportion of about 1 to about 20%, preferably of about 5 to about 15% (percentages as weight by volume=W/V). The liquid carrier vehicle comprises a pharmaceutically or veterinary acceptable organic solvent and optionally an organic cosolvent.

An especially preferred embodiment is spot-on formulation comprising a compound of formula (II) or a salt thereof, with spot-on formulations comprising the thioamide derivative fipronil being most especially preferred.

Also contemplated are the pharmaceutically or veterinary acceptable acid or base salts, where applicable, of the active compounds provided for herein. The term “acid” contemplates all pharmaceutically or veterinary acceptable inorganic or organic acids. Inorganic acids include mineral acids such as hydrohalic acids, such as hydrobromic and hydrochloric acids, sulfuric acids, phosphoric acids and nitric acids. Organic acids include all pharmaceutically or veterinary acceptable aliphatic, alicyclic and aromatic carboxylic acids, dicarboxylic acids tricarboxylic acids and fatty acids. Preferred acids are straight chain or branched, saturated or unsaturated C₁-C₂₀ aliphatic carboxylic acids, which are optionally substituted by halogen or by hydroxyl groups, or C₆-C₁₂ aromatic carboxylic acids. Examples of such acids are carbonic acid, formic acid, fumaric acid, acetic acid, propionic acid, isopropionic acid, valeric acid, &-hydroxy acids, such as glycolic acid and lactic acid, chloroacetic acid, benzoic acid, methane sulfonic acid, and salicylic acid. Examples of dicarboxylic acids include oxalic acid, malic acid, succinic acid, tataric acid and maleic acid. An example of a tricarboxylic acid is citric acid. Fatty acids include all pharmaceutically or veterinary acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

The term “base” contemplates all pharmaceutically or veterinary acceptable inorganic or organic bases. Such bases include, for example, the alkali metal and alkaline earth metal salts, such as the lithium, sodium, potassium, magnesium or calcium salts. Organic bases include the common hydrocarbyl and heterocyclic amine salts, which include, for example, the morpholine and piperidine salts.

The organic solvent for the liquid carrier vehicle will preferably have a dielectric constant of between about 10 and about 35, preferably between about 20 and about 30, the content of this solvent in the overall composition preferably representing the remainder to 100% of the composition. It is well within the skill level of the practitioner to select a suitable solvent on the basis of these parameters.

The organic cosolvent for the liquid carrier vehicle will preferably have a boiling point of less than about 100!C., preferably of less than about 80!C., and will have a dielectric constant of between about 10 and about 40, preferably between about 20 and about 30; this cosolvent can advantageously be present in the composition according to a weight/weight (W/W) ratio with respect to the solvent of between about 1/15 and about ½; the cosolvent is volatile in order to act in particular as drying promoter and is miscible with water and/or with the solvent. Again, it is well within the skill level of the practitioner to select a suitable solvent on the basis of these parameters.

The organic solvent for the liquid carrier includes the commonly acceptable organic solvents known in the formulation art. These solvents may be found, for example, in Remington Pharmaceutical Science, 16^{th} Edition (1986). These solvents include, for example, acetone, ethyl acetate, methanol, ethanol, isopropanol, dimethylformamide, dichloromethane or diethylene glycol monoethyl ether (Transcutol). These solvents can be supplemented by various excipients according to the nature of the desired phases, such as C₈-C₁₀ caprylic/capric triglyceride (Estasan or Miglyol 812), oleic acid or propylene glycol.

The liquid carrier may also comprise a microemulsion. Microemulsions are also well suited as the liquid carrier vehicle. Microemulsions are quaternary systems comprising an aqueous phase, an oily phase, a surfactant and a cosurfactant They are translucent and isotropic liquids.

Microemulsions are composed of stable dispersions of microdroplets of the aqueous phase in the oily phase or conversely of microdroplets of the oily phase in the aqueous phase. The size of these microdroplets is less than 200 nm (1000 to 100,000 nm for emulsions). The interfacial film is composed of an alternation of surface-active (SA) and co-surface-active (Co-SA) molecules which, by lowering the interfacial tension, allows the microemulsion to be formed spontaneously.

The oily phase can in particular be formed from mineral or vegetable oils, from unsaturated polyglycosylated glycerides or from triglycerides, or alternatively from mixtures of such compounds. The oily phase preferably comprises triglycerides and more preferably medium-chain triglycerides, for example C₈-C₁₀ caprylic/capric triglyceride. The oily phase will represent, in particular, from about 2 to about 15%, more particularly from about 7 to about 10%, preferably from about 8 to about 9%, V/V of the microemulsion.

The aqueous phase includes, for example water or glycol derivatives, such as propylene glycol, glycol ethers, polyethylene glycols or glycerol. Propylene glycol, diethylene glycol monoethyl ether and dipropylene glycol monoethyl ether are especially preferred. Generally, the aqueous phase will represent a proportion from about 1 to about 4% V/V in the microemulsion.

Surfactants for the microemulsion include diethylene glycol monoethyl ether, dipropyelene glycol monomethyl ether, polyglycolysed C₈-C₁₀ glycerides or polyglyceryl-6 dioleate. In addition to these surfactants, the cosurfactants include short-chain alcohols, such as ethanol and propanol.

Some compounds are common to the three components discussed above, i.e., aqueous phase, surfactant and cosurfactant. However, it is well within the skill level of the practitioner to use different compounds for each component of the same formulation.

The cosurfactant to surfactant ratio will preferably be from about 1/7 to about ½. There will preferably be from about 25 to about 75% V/V of surfactant and from about 10 to about 55% V/V of cosurfactant in the microemulsion.

Likewise, the co-solvents are also well known to a practitioner in the formulation art. Preferred co-solvents are those which is a promoter of drying and include, for example, ethanol, absolute ethanol, isopropanol (2-propanol) or methanol.

The crystallization inhibitor can in particular be present in a proportion of about 1 to about 20% (W/W), preferably of about 5 to about 15%. The inhibitor preferably corresponds to the test in which 0.3 ml of a solution comprising 10% (W/V) of the compound of formula (I) in the liquid carrier and 10% of the inhibitor are deposited on a glass slide at 20° C. and allowed to stand for 24 hours. The slide is then observed with the naked eye. Acceptable inhibitors are those whose addition provides for few or no crystals, and in particular less than 10 crystals, preferably 0 crystals.

Although this is not preferred, the formulation can optionally comprise water, in particular in a proportion of 0 to about 30% (volume by volume V/V), in particular of 0 to about 5%.

The formulation can also comprise an antioxidizing agent intended to inhibit oxidation in air, this agent being in particular present in a proportion of about 0.005 to about 1% (W/V), preferably of about 0.01 to about 0.05%.

Crystallization inhibitors which can be used in the invention include:
polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others,
anionic surfactants, such as alkaline stearates, in particular sodium, potassium or ammonium stearate; calcium stearate or triethanolamine stearate; sodium abietate; alkyl sulphates, in particular sodium lauryl sulphate and sodium cetyl sulphate; sodium dodecylbenzenesulphonate or sodium dioctyl sulphosuccinate; or fatty acids, in particular those derived from coconut oil,
cationic surfactants, such as water-soluble quaternary ammonium salts of formula N⁺R′R″R′″R″″Y⁻, in which the R radicals are identical or different optionally hydroxylated hydrocarbon radicals and Y⁻is an anion of a strong acid, such as halide, sulphate and sulphonate anions; cetyltrimethylammonium bromide is one of the cationic surfactants which can be used,
amine salts of formula N⁺R′R″R′″, in which the R radicals are identical or different optionally hydroxylated hydrocarbon radicals; octadecylamine hydrochloride is one of the cationic surfactants which can be used,
non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide,
amphoteric surfactants, such as substituted lauryl compounds of betaine,
or preferably a mixture of at least two of the compounds listed above.

In a particularly preferred embodiment, a crystallization inhibitor pair will be used. Such pairs include, for example, the combination of a film-forming agent of polymeric type and of a surface-active agent. These agents will be selected in particular from the compounds mentioned above as crystallization inhibitor.

Particularly preferred film-forming agents of polymeric type include:
the various grades of polyvinylpyrrolidone,
polyvinyl alcohols, and
copolymers of vinyl acetate and of vinylpyrrolidone.

Especially preferred surface-active agents, include those made of non-ionic surfactants, preferably polyoxyethylenated esters of sorbitan and in particular the various grades of polysorbate, for example Polysorbate 80.

The film-forming agent and the surface-active agent can in particular be incorporated in similar or identical amounts within the limit of the total amounts of crystallization inhibitor mentioned elsewhere.

The pair thus constituted secures, in a noteworthy way, the objectives of absence of crystallization on the coat and of maintenance of the cosmetic appearance of the fur, that is to say without a tendency towards sticking or towards a sticky appearance, despite the high concentration of active material.

Particularly preferred antioxidizing agents are those conventional in the art and include, for example, butylated hydroxyanisole, butylated hydroxytoluene, ascorbic acid, sodium metabisulphite, propyl gallate, sodium thiosulphate or a mixture of not more than two of them.

The formulation adjuvants discussed above are well known to the practitioner in this art and may be obtained commercially or through known techniques. These concentrated compositions are generally prepared by simple mixing of the constituents as defined above; advantageously, the starting point is to mix the active material in the main solvent and then the other ingredients or adjuvants are added.

The volume applied can be of the order of about 0.3 to about 1 ml, preferably of the order of about 0.5 ml, for cats and of the order of about 0.3 to about 5 ml for dogs, depending on the weight of the animal.

The pour-on solutions according to the invention, which are advantageously oily, generally comprise a diluent or vehicle and also a solvent (organic solvent) for the compound of formula (II) if the latter is not soluble in the diluent. Low concentrations of from 0.05 to 10% weight volume, more particularly from 0.1 to 2%, are preferred. Optimally, the value is between 0.25 and 1.5%; in particular in the region of 1%.

Organic solvents which can be used in the inventive pour-on solutions, mention may be made in particular of: acetyltributyl citrate, fatty acid esters such as the dimethyl ester, diisobutyl adipate, acetone, acetonitrile, benzyl alcohol, butyl diglycol, dimethylacetamide, dimethylformamide, dipropylene glycol n-butyl ether, ethanol, isopropanol, methanol, diethylene glycol monoethyl ether, diethylene glycol monomethyl ether, monomethylacetamide, dipropylene glycol monomethyl ether, liquid polyoxyethylene glycols, propylene glycol, 2-pyrrolidone, acetyl tributyl citrate, in particular N-methylpyrrolidone, diethylene glycol monoethyl ether, ethylene glycol and diethyl phthalate, or a mixture of at least two of these solvents.

As vehicle or diluent for the inventive pour-on solutions, mention may be made in particular of:

plant oils such as soybean oil, groundnut oil, castor oil, corn oil, cotton oil, olive oil, grape seed oil, sunflower oil, etc.; mineral oils such as petrolatum, paraffin, silicone, etc.; aliphatic or cyclic hydrocarbons or alternatively, for example, medium-chain (C₈ to C₁₂ in particular) triglycerides:

An emollient and/or spreading and/or film-forming agent will preferably be added, this agent being selected in particular from:

polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, polyoxypropylene 15 stearyl ether, sodium carboxymethylcellulose, silicone oils, polydiorganosiloxane oils, in particular polydimethylsiloxane (PDMS) oils, for example those containing silanol functionalities, or a 45V2 oil,

anionic surfactants such as alkaline stearates, in particular sodium, potassium or ammonium stearates; calcium stearate, triethanolamine stearate; sodium abietate; alkyl sulphates, in particular sodium lauryl sulphate and sodium cetyl sulphate; sodium dodecylbenzenesulphonate, sodium dioctylsulphosuccinate; fatty acids, in particular those derived from coconut oil,

cationic surfactants such as water-soluble quaternary ammonium salts of formula N⁺R′R″R′″R″″, Y⁻ in which the radicals R are optionally hydroxylated hydrocarbon radicals and Y⁻ is an anion of a strong acid such as the halide, sulphate and sulphonate anions; cetyltrimethylammonium bromide is among the cationic surfactants which can be used,

amine salts of formula N⁺R′R″R′″ in which the radicals R are optionally hydroxylated hydrocarbon radicals; octadecylamine hydrochloride is among the cationic surfactants which can be used,

nonionic surfactants such as sorbitan esters, which are optionally polyoxyethylenated, in particular polysorbate 80, polyoxyethylenated alkyl ethers; polyoxypropylated fatty alcohols such as polyoxypropylene-styrol ether; polyethylene glycol stearate, polyoxyethylenated derivatives of castor oil, polyglycerol esters, polyoxyethylenated fatty alcohols, polyoxyethylenated fatty acids, copolymers of ethylene oxide and propylene oxide,

amphoteric surfactants such as the substituted lauryl compounds of betaine;

or a mixture of at least two of these agents.

The solvent will be used in proportion with the concentration of the compound II and its solubility in this solvent.

The emollient is preferably used in a proportion of from about 0.1 to about 10%, in particular from about 0.25 to about 5%, by volume.

An especially preferred compound (I) is derivative of formula (III):

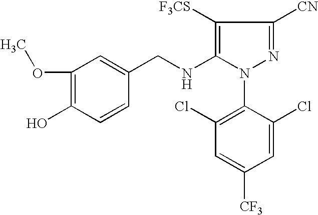

The formulations according to the invention are extremely effective for long durations of time in the treatment of parasites such as fleas of mammals and, in particular, of small mammals such as dogs and cats. The inventive formulations exhibit a degree of effectiveness against other parasitic insects and in particular fleas, ticks, mites, mosquitoes and flies.

The subject of the present invention is also a process for the elimination of parasites in animals, in particular fleas and ticks in companion animals and Boophilus microplus, from cattle and sheep using a direct pour-on or spot-on skin solution according to the present invention, so as obtain long-lasting and broad-spectrum efficacy. According to a first embodiment, the process consists in applying the solution, the application preferably being repeated every month, preferably every two months.

According to a second embodiment for livestock, the process consists of applying the solution to the animals in pastures and/or before they arrive in pasture or consists of in applying the solution to the animals before they arrive in the “feed lot”, it being possible for this application to be the final one before the animals are slaughtered. Obviously, the process may also consist in combining these procedures, namely the first followed by the second.

For livestock, the efficacy advantageously makes it possible to stop any application about 1 to about 3 months before slaughter, in particular between about 1.5 and about 2.5 months, more particularly about two months before slaughter.

The solutions according to the invention may be applied using any means known per se, preferably using an applicator gun or a metering flask.

An aim of the method is not therapeutic and is, in particular, to cleanse the skin and the hairs of the animals by eliminating the parasites, which are present thereon, as well as their residues and dejections. The result of this is that the animals are no longer stressed by the parasites and their bites, this having positive consequences, for example on their growth and on the use of their food ration.

A further method provided for in the present invention is a method for the control or elimination of external parasites from an animal comprising topically applying at least monthly to an animal a parasitically effective amount of a spray formulation comprising a compound of formula (II). Spray formulations comprise of the active parasiticide ingredient combined with vehicles, diluents, crystallization inhibitors, film forming agents and others. These include alcohols, such as isopropyl, ethyl, methyl alcohols, among others, as well polyvinylpyrrolidone, polyvinyl alcohols, copolymers of vinyl acetate and vinylpyrrolidone, polyethylene glycols, benzyl alcohol, mannitol, glycerol, sorbitol, polyoxyethylenated sorbitan esters; lecithin, polyoxypropylene 15 stearyl ether, sodium carboxymethylcellulose, silicone oils, polydiorganosiloxane oils, in particular polydimethylsiloxane (PDMS) oils, for example those containing silanol functionalities, or a 45V2 oil.

Another subject of the invention is a therapeutic method using the external device according to the invention, intended for the treatment and prevention of parasitoses having pathogenic consequences.

The subject of the present invention is also the use of the compounds II, which in turn degrade N-1-arylpyrazoles, in particular compound of formula I and most particular fipronil, while on the animal's skin as well as for the manufacture of a direct pour-on or spot-on skin solution comprising the compound (II) in a low volume and designed to release the compound (II) onto the skin and the hairs of an animal contact action against the parasites that affect the animal, such as companion animals, cattle, sheep, in particular ticks in dogs, cats, and cattle, such as Boophilus microplus, Rhipicephalus sanguineus, Dermacentor sp, Amblyomma sp, Ixodes sp, Haemaphysalis, sp as well as fleas (Clenocephalides felis) and sheep blowfly and lice.

The use according to the invention is directed towards producing skin solutions as described above or oral formulations.

Applicants discovered thioamide derivatives of 1-N-aryl pyrazoles will degrade in the presence of heat UV or fluorescent light to the corresponding cyano-containing derivative. The cyano derivative was not formed in the presence of acid, base or oxidizing agents. Additionally, the sulfone derivative was formed over time. These derivatives were formed in an amount that was sufficient to combat fleas and ticks in the animal. In view of this compounds of formula II may be administered topically or orally to an animal, where they will slowly convert to the cyano or sulfone derivatives, which also have activity against fleas and ticks.

Preferred oral formulations include a chewable veterinary formulation, which does not contain animal products, which comprises:
effective amount of at least one compound of formula II;
at least one filler;
at least one disintegrant;
at least one non-animal product containing flavor or flavor derived from a non-animal source;
at least one binder;
at least one humectant;
at least one granulating solvent; and
optionally, at least one antioxidant, at least one buffering agent, at least one preservative, or at least one colorant;
   or, more preferably, a chewable veterinary formulation, which does not contain animal products, which comprises:
effective amount of at least one compound of formula II;
a filler selected from the group consisting of soy protein, corn cob, or corn gluten meal;
disintegrant;
a non-animal product containing flavor or a flavor derived from non-animal source which is a hickory smoke flavor;
a binder;
humectant;
granulating solvent; and
optionally, an antioxidant, a buffering agent, preservative, or a colorant.

Most preferred are chewable veterinary formulations, which do not contain animal products, which comprise:
an effective amount of at least one compound of formula II
about 20 to about 60% of a filler selected from the group consisting of soy protein, corn cob, or corn gluten meal;
about 1 to about 20% of a disintegrant;
about 0.1 to about 20% of a non-animal product containing flavor; or a flavor derived from a non-animal source
about 0.5 to 10% a binder;
about 5 to about 20% of a humectant; and
about 5 to about 20% granulating solvent, based upon total weight of formulation.

Especially preferred are chewable veterinary formulations, which do not contain animal products which comprise:
an effective amount of at least one compound of formula II;
about 20 to about 60% of a filler selected from the group consisting of soy protein, corn cob, or corn gluten meal;
about 1 to about 20% of a disintegrant;
about 0.1 to about 20% of the non-animal product containing flavor or flavor derived from a non-animal source is a hickory barbecue flavor;
about 0.5 to 10% a binder;
about 5 to about 20% of a humectant; and
about 5 to about 20% granulating solvent, and, optionally
about 0.05% to about 1.0% of an antioxidant,
about 0.05 to about 1.0% of a preservative, and
about 0.001 to about 10% of a colorant, based upon total weight of formulation.

Another preferred embodiment is a tablet, which does not contain animal products, which comprises:
an effective amount of at least one compound of formula II
at least one filler;
at least one non-animal product containing flavor or flavor derived from a non-animal source;
at least one lubricant;
at least one flow aid; and
optionally, at least one antioxidant, at least one pH modifier, at least one binder, at least one disintegrant, at least one surfactant, at least one preservative, and at least one colorant, and is optionally coated with at least one coating.

An important feature of the present invention is the flavor that does not contain animal products or is not derived from an animal source. Flavors derived from catnip, the valarian plant or fruit are not contemplated by the present invention. Flavors include those known in pet foods which are artificial and include, for example:

| | |
|---|---|
| DRY GARLIC-ADE OS | Formulated to provide a pungent garlic aroma. |
| LIQUID GARLIC-ADE OS | Same as dry garlic-ade in an oil miscible liquid |
| | form. |
| LIQUID GARLIC-ADE CONCENTRATE OM | Same as Dry Garlic-Ade but in a concentrated, |
| | oil miscible liquid form. |
| DRY ONION-ADE | Formulated to deliver an aroma and taste of |
| | cooked onions. |
| DRY GARLIC ONION-ADE | A dry blend of Garlic-Ade and Onion-Ade. |
| DRY CHEESE-ADE | A strong cheddar cheese flavor and aroma. |
| LIQUID CHEESE-ADE OM | An oil miscible, liquid version of Dry Cheese- |
| | Ade. |
| DRY CHICKEN-ADE | Formulated to provide the aroma of baked |
| | chicken. |
| LIQUID CHICKEN-ADE OS | An oil soluble liquid version of Dry Chicken- |
| | Ade. |
| LIQUID CHICKEN-ADE OS CONCENTRATE | A concentrated form of Liquid Chicken-Ade OS. |
| FFA | |
| DRY LIVER-ADE | Formulated to provide the aroma and flavor of |
| | cooked liver. |
| LIQUID LIVER-ADE CONCENTRATE | A concentrated liquid version of Dry Liver-Ade. |
| DRY PET-ADE BEEF STEW | A blend of many flavor components which |
| | provide of beef stew. |
| LIQUID PET-ADE BEEF STEW OS | An oil soluble, liquid version of Dry Pet-Ade |
| | Beef Stew. |
| PET-ADE BEEF STEW CONCENTRATE | A concentrated liquid version of Dry Pet-Ade |
| | Beef Stew. |
| DRY BEEF-ADE | A dry flavor formulated to provide the appeal of |
| | a baking roast. |
| DRY FISH MEAL FLAVOR CONCENTRATE | A dry flavor formulated to provide the odor of |
| | fish meal. |
| LIQUID FISH MEAL FLAVOR | A liquid version of Dry Fish Meal Flavor. |
| CONCENTRATE | |
| DRY KANIN-KRAVE | A spicy bone marrow flavor. |
| DRY BACON-ADE | A dry flavor which provides the aroma of frying |
| | bacon. |

Sources for these flavors are well-know to a practitioner in this art. For example, Kermine Petfood Nutrisurance is a vegetarian flavor for pet food is sold by Kemine industries, Inc., Des Moines, Iowa. A discussion of commercial smoke flavorings is provided by Guillen et al. in J. Agr. and Food Chemistry vol. 4.

Preferred are the GRILLIN' line of grill flavors and blends marketed by the Red Arrow Products Company, LLC, Manitowoc, Wis. for human and pet food. These include GRILLIN' TYPE CB-200, GRILLIN' TYPE SD, GRILLIN' TYPE WS-50, GRILLIN' TYPE CN, GRILLIN' TYPE CB, GRILLIN' TYPE GS and GRILLIN' TYPE NBF.

Especially preferred are hickory smoked flavoring produced by combining torula yeast and an aqueous hickory smoke solution, sold by Red Arrow Products Co. as CHARTOR HICKORY or a hickory smoke flavoring produced by combining maltodextin with an aqueous hickory smoke solution, sold by Red Arrow Products Co. as CHARDEX HICKORY. Other flavors contemplated by the invention include those which impart a natural dry smoke flavor. These include CHARZYME (a smoke flavor produced by combining barley malt flour with an aqueous smoke flavor), CHARMAIZE (a smoke flavor produced by combining yellow flower and an aqueous smoke flavor) and CHARSALT (a blend of dendritic salt, aqueous smoke flavor, and dydrated silicon dioxide. All of these flavors may be obtained by Red Arrow Products Co.

The determination of the amounts of flavor for a particular product is easily determined by a practitioner of this art. Typical ranges are from up to about 10%. Also preferred are those flavors which provide a savory flavor. These flavors are well known to a practitioner of this art.

Absorbents may also be added to the inventive formulations. Such compounds are well known in the art to the practitioner as well as their use in pastes. These compounds effectively prevents or alleviates the phase separation of the product during storage. Preferred absorbents include magnesium carbonate, calcium carbonate, potassium bicarbonate, sodium bicarbonate, starch, cellulose and its derivatives, or mixtures of absorbents, with magnesium carbonate being especially preferred. The inclusion of these compounds is optional with amounts of 0% to about 30%, 0 to about 15% or about 1% to about 15% or about 1% to about 10%, based on total weight of the formulation being especially preferred.

Additionally, the inventive formulations may contain other inert ingredients such as antioxidants, preservatives, stabilizers or surfactants. These compounds are well known in the formulation art. Antioxidant such as an alpha tocopheral, ascorbic acid, ascrobyl palmitate, fumeric acid, malic acid, sodium ascorbate, sodium metabisulfate, n-propyl gallate, BHA (butylated hydroxy anisole), BHT (butylated hydroxy toluene) monothioglycerol and the like, may be added to the present formulation. The antioxidants are generally added to the formulation in amounts of from about 0.01 to about 2.0%, based upon total weight of the formulation, with about 0.1 to about 1.0% being especially preferred. Preservatives, such as the parabens (methylparaben and/or propylparaben), are suitably used in the formulation in amounts ranging from about 0.01 to about 2.0%, with about 0.05 to about 1.0% being especially preferred. Other preservatives include benzalkonium chloride, benzethonium chloride, benzoic acid, benzyl alcohol, bronopol, butylparaben, cetrimide, chlorhexidine, chlorobutanol, chlorocresol, cresol, ethylparaben, imidurea, methylparaben, phenol, phenoxyethanol, phenylethyl alcohol, phenylmercuric acetate, phenylmercuric borate, phenylmercuric nitrate, potassium sorbate, sodium benzoate, sodium propionate, sorbic acid, thimerosal, propyl paraben, myristyl gama-picolinium chloride, paraben methyl, paraben propyl and quaternary ammonium compounds and the like.

Surfactants in amounts from about 0.001 to about 1%, based upon total weight may also be added to help solubilize the active drug, to prevent crystallization, and to prevent phase separation. Some examples of the surfactants are: glyceryl monooleate, polyoxyethylene sorbitan fatty acid esters, sorbitan esters, polyvinyl alcohol, Pluronics, polysorbate 80, sodium lauryl sulfate, poloxomers (LUTROL F87), etc. Again, these compounds, as well as their amounts are well known in the art.

Colorants may be added to the inventive formulations. Colorants contemplated by the present invention are those commonly known in the art. Specific colorants include, for example, dyes, an aluminum lake, caramel (which may also function as a flavor), colorant based upon iron oxide or a mixture of any of the foregoing. Especially preferred are organic dyes and titanium dioxide. Preferred ranges include from about 0.5% to about 25%.

The chewable formulations provided for in the invention may also- include lubricants, such as polyethylene glycols (PEG's or CARBOWAX), corn oil, mineral oil, hydrogenated vegetable oils (STEROTEX OR LUBRITAB), peanut oil and/or castor oil. The inclusion and identity of a lubricant is readily determined by a practitioner of this art are present in amounts, for example, of about 0.01 to about 20%, based upon total weight in the composition.

Compounds which stabilize the pH of the formulation (pH modifiers) are also contemplated. Again, such compounds are well known to a practitioner in the art as well as how to use these compounds. Buffering systems include, for example, systems selected from the group consisting of acetic acid/acetate, malic acid/malate, citric acid/citrate, tataric acid/tartrate, lactic acid/lactate, phosphoric acid/phosphate, glycine/glycimate, tris, glutamic acid/glutamates and sodium carbonate. Preferred ranges for pH include from about 4 to about 6.5.

Other compounds contemplated by the inventive formulations include complexing agents, such as cyclodextrins, PVP, PEG, ethyl lactate and niacinamide. Amounts of such compounds to be included in the inventive formulation are well known to a practitioner of the art. Also contemplated are therapeutic agents to be in the form of emulsions, liposomes or micelles.

The inventive formulation may be administered to a warm-blooded animals, such as cattle, sheep, goats, pigs, cats, dogs, horses, llamas, deer, rabbits, skunks, raccoons, camels, humans and the like, or birds. The amount of compound of formula II depends on the specific compound, the animal being treated, the disease state, and the severity of the disease state. The determination of those factors is well within the skill level of the practitioner. Generally, such preparation normally contain about 0.0005 to about 50% of compound formula II by total weight of composition. Preferred formulations are those containing about 0.01 to about 10% of compound formula II and especially preferred formulations are those containing about 2.5 to about 5% of compound formula II. Other preferred amounts include about 0.1 to about 0.01 to about 50% or about 10% or about 0.5 to about 3%.

This invention further provides for tablets that do not contain animal products which comprise, in addition to the non-animal product containing flavor or flavor derived from a non-animal source, at least one compound of formula II, flavor, filler, lubricant, and flow aid. Optionally, the inventive tablets may further contain at least one of the following ingredients: colorants, binders, antioxidants, disintegrants, or preservatives. Moreover, in an alternative embodiment this invention provides for tablets which are coated. The inventive tablets are prepared according to methods conventional in the art, such as wet and dry granulation processes.

Many of the ingredients for the tablet include those provided for in the chewable formulations. With respect to fillers (or diluents), the inventive tablets contemplate all the fillers which are known in the tablet art. Non-limiting examples of fillers include anhydrous lactose, hydrated lactose, sprayed dried lactose, crystalline maltose and maltodextrins.

Flow aids or glidants are also well known in the art and include, for example, silicon dioxide (CARBOSIL) or silica gel (SYLOID), talc, starch, calcium, stearate, magnesium stearate, and aluminum magnesium silicate (NEUSILIN). Amounts of flow aids are readily determined by a practitioner in this art and include for using about 0.01 to about 25%, based upon weight of total composition. Non-limiting examples of lubricants for the tablets include magnesium and calcium stearate and stearic acid. Again, the various lubricants are well known to a practitioner of this art as well as the amounts of these compounds. Ranges include from about 0.01 to about 20%.

The tablets provided for by this invention may be coated using techniques conventional in the art. Coatings include sugar coatings, such as seal coatings, subcoatings, and syrup coatings, as well as film coatings, such as pan-pour coatings and pan spray coatings. As well known to a practitioner of this art, the coatings contain additional components such as solvents, plasticizers, colorants, opaquant-extenders and film formers.

This invention also provides for a premix formulation that comprises an effective amount of at least one compound of formula (II). A premix formulation is a formulation that is mixed into the animal's food either every time the animal is fed or daily. Premix formulations comprise the active parasiticide ingredient(s) and one or more ingredients that provide flavor and protects the stability of the active ingredient. These include vegetable farinaceous meals, such as soybean, grain, corn, sorgham and others as well as pH stabilizers.

Also, contemplated by the inventive methods or formulations are formulations that contain an additional active parasitical ingredient, such as an insecticide, acaricide, parasiticide, etc. These compounds include avermectin and milibemycins. The avermectin and milbemycin series of compounds are potent anthelmintic and antiparasitic agents against a wide range of internal and external parasites. The compounds which belong to this series are either natural products or are semi-synthetic derivatives thereof. The structure of these two series of compounds are closely related and they both share a complex 16-membered macrocyclic lactone ring; however, the milbemycin do not contain the aglycone substituent in the 13-position of the lactone ring; examples include milbemectin and moxidectin. The natural product avermectins are disclosed in U.S. Pat. No. 4,310,519 to Albers-Schonberg, et al., and the 22,23-dihydro avermectin compounds are disclosed in Chabala, et al., U.S. Pat. No. 4,199,569. For a general discussion of avermectins, which include a discussion of their uses in humans and animals, see “Ivermectin and Abamectin,” W.C. Campbell, ed., Springer-Verlag, New York (1989). Naturally occurring milbemycins are described in Aoki et al., U.S. Pat. No. 3,950,360 as well as in the various references cited in “The Merck Index” 12^{th} ed., S. Budavari, Ed., Merck & Co., Inc. Whitehouse Station, N.J. (1996). Semisynthetic derivatives of these classes of compounds are well known in the art and are described, for example, in U.S. Pat. No. 5,077,308, U.S. Pat. No. 4,859,657, U.S. Pat. No. 4,963,582, U.S. Pat. No. 4,855,317, U.S. Pat. No. 4,871,719, U.S. Pat. No. 4,874,749, U.S. Pat. No. 4,427,663, U.S. Pat. No. 4,310,519, U.S. Pat. No. 4,199,569, U.S. Pat. No. 5,055,596, U.S. Pat. No. 4,973,711, U.S. Pat. No. 4,978,677, and U.S. Pat. No. 4,920,148. Especially preferred compounds include ivermectin, abamectin, doramectin, emamectin, eprinomectin, latidectin, lepimectin and selamectin.

Another class of compounds that may be included in the inventive formulations or methods is insect growth regulators (IGR). Compounds belonging to this group are well known to the practitioner and represent a wide range of different chemical classes. These compounds all act by interfering with the development or growth of the insect pests. Compounds with an ovicidal and/or larvicidal effect on the immature stages of various ectoparasites are already known, for example from U.S. Pat. No. 5,439,924. Among these compounds described are those IGR compounds which act either by blocking the development of the immature stages (eggs and larvae) into adult stages, or by inhibiting the synthesis of chitin. Insect growth regulators are described, for example, in U.S. Pat. No. 3,748,356; U.S. Pat. No. 3,818,047; U.S. Pat. No. 4,225,598; U.S. P atent 4,798,837; and U.S. Pat. No. 4,751,225, as well as in EP 179,022 or U.K. 2,140,010. French Patent No. A-2,713,889 which generally describes an IGR combination comprising at least one compound with juvenile hormone activity and chitin synthesis inhibitors, with at least one of three N-aryldiazole compounds, in particular fipronil, to control many harmful insects belonging to very varied orders.

Examples of IGR which may be used in the formulation of the present invention include compounds which mimic juvenile hormones, in particular:
Azadirchtin-Agridyne
Diofenolan (Ciba Geigy)
Fenoxycarb (Ciba Geigy)
Hydroprene (Sandoz)
Kinoprene (Sandoz)
Methoprene (Sandoz)
Pyriproxyfen (Sumitomo/Mgk)
Tetrahydroazadirachtin (Agridyne)
4-chloro-2-(2-chloro-2-methylpropyl)-5-(6-iodo-3-pyridylmethoxy)pyridizin-3(2H)-one
   and chitin-synthesis inhibitors, in particular:
chlorfluazuron (Ishihara Sangyo)
cyromazine (Ciba Geigy)
diflubenzuron (Solvay Duphar)
fluazuron (Ciba Geigy)
flucycloxuron (Solvay Duphar)
flufenoxuron (Cyanamid)
hexaflumuron (Dow Elanco)
lufenuron (Ciba Geigy)
tebufenozide (Rohm & Haas)
teflubenzuron (Cyanamid)
triflumuron (Bayer)
   these compounds being defined by their international common name (The Pesticide Manual, 10^{th} edition, 1994, Ed. Clive Tomlin, Great Britain).

Chitin-synthesis inhibitors also include compounds such as 1-(2,6-difluorobenzoyl)-3-(2-fluoro4-((trifluoromethyl) phenylurea, 1-(2,6-difluorobenzoyl)-3-(2-fluoro-4-(1,1,2,2-tetrafluoroethoxy)phenylurea and 1-(2,6-difluorobenzoyl)-3-(2-fluoro-4-trifluoromethyl)phenylurea. Novaluron (Isagro, Italian company) is also an example of an IGR.

Especially preferred IGR include methoprenes, pyriproxyfens, hydroprene, cyromazine, lufenuron, 1-(2,6-difluorobenzoyl)-3-(2-fluoro-4-(trifluoromethyl)phenylurea and novaluron.

Also contemplated are, for example, nodulisporic acid or nodulisporic acid derivatives. Nodulisporic acid and nodulisporic acid derivatives are known in the art as a class of compounds that are potent endo- and ectoantiparasitic agents. These compounds are based upon three structures, A, B or C, which have the following structures:

nodulisporic acid (compound A)

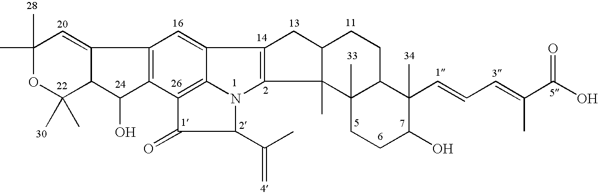

29,30-dihydro-20,30-oxa-nodulisporic acid (compound B) 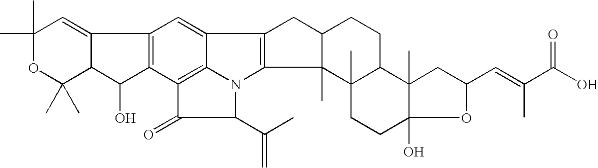
and

31-hydroxy-20,30-oxa-29,30,31,32-tetrahydro-nodulisporic acid (compound C) 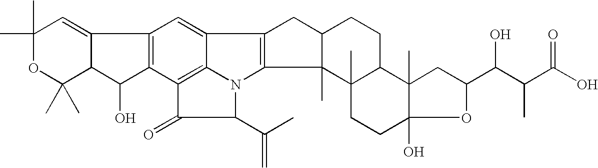
These compounds were obtained from the fermentation culture of Nodulisporium sp. MF-5954 (ATCC 74245) and the isolation and purification of the three nodulisporic acids are disclosed in U.S. Pat. No. 5,399,582. Derivatives of these compounds are described in WO 96/29073 and U.S. Pat. Nos. 5,945,317; 5,962,499; 5,834,260; 6,399,796; 6,221,894; 6,136,838; 5,595,991; 5,299,582; and 5,614,546.

Nodulisporic acid derivatives possess potent activity against parasites, particularly ectoparasites, insects, and acarides, infecting man, animals and plants. These compounds have utility in human and animal health, agriculture and pest control in household and commercial areas.

Non-limiting examples of household pests are cockroach, Blazella sp., clothes moth, Tineola sp., carpet beetle, Attagenus sp., the housefly Musca domestica as well as fleas, house dust mites, termites and ants.

Examples of insect pests of stored grains are Tribolium sp., Tenebrio sp. and of agricultural plants are aphids, (Acyrthiosiphon sp.); against migratory orthopterans are locusts and immature stages of insects living on plant tissue. The compounds are also highly useful in treating acreage infested with fire ants and nests. The compounds are scattered above the infested area in low levels in bait formulations which are brought back to the nest. In addition to a direct-but-slow onset toxic effect on the fire ants, the compound has a long-term effect on the nest by sterilizing the queen which effectively destroys the nest.

Nodulisporic acid and its derivatives are also effective against arthropod and insect pests, for example fleas, ticks, lice and other biting insects in domesticated animals and poultry, such as Ctenophalides, Rhipicephalus, Dermacentor, Amblyomma, Ixodes, Psoroptes, Lucilia and Hematobia.

This invention includes all nodulisporic acid derivatives know in the art, including all steroisomers, such as those described in the prior publications described above, which are expressly incorporated by reference. Especially preferred are spot-on formulations comprising nordulisporic acid derivatives of the formula: 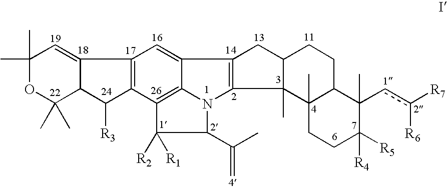
wherein
R₁ is
   (1) hydrogen,
   (2) optionally substituted alkyl,
   (3) optionally substituted alkenyl,
   (4) optionally substituted alkynyl,
   (5) optionally substituted cycloalkyl,
   (6) optionally substituted cycloalkenyl,
   where the substituents on the alkyl, alkenyl, alkynyl, cycloalkyl and cycloalkenyl are 1 to 3 groups independently selected from
      (i) alkyl,
      (ii) alkyl, where X is O or S(O)ₘ.
      (iii) cycloalkyl,
      (iv) hydroxy,
      (v) halogen,
      (vi) cyano,
      (vii) carboxy,
      (viii) NY¹Y², where Y¹ and y² are independently H or alkyl,
      (ix) alkanoylamino, preferably and
      (x) aroylamino, preferably wherein said aroyl is optionally substituted with 1 to 3 groups independently selected from R^{f}
   (7) aryl or arylalkyl, wherein said aryl is optionally substituted with 1 to 3 groups independently selected from R^{f},
   (8) perfluoroalkyl,
   (9) a 5- or 6-member heterocycle containing from 1 to 4 heteroatoms independently selected from oxygen, sulfur and nitrogen atoms optionally substituted by 1 to 3 groups independently selected from hydroxy, oxo, alkyl, and halogen, and which may be saturated or partly unsaturated,
R₂, R₃, and R₄ are independently OR^{a}, OCO₂R^{b}, OC(O)NR^{c}R^{d}; or
R₁ and R₂ represent ═O, ═NOR^{a} or ═N—NR^{c}R^{d};
R₅ and R₆ are H; or
R₅ and R₆ together represent —O—;
R₇ is
   (1) CHO, or
   (2) the fragment 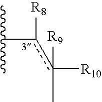
R₈ is
   (1) H,
   (2) OR^{a}, or
   (3) NR^{c}R^{d}
R₉ is
   (1) H, or
   (2) OR^{a};
R₁₀ is
   (1) CN,
   (2) C(O)OR^{b},
   (3) C(O)N(OR^{b})R^{c},
   (4) C(O)NR^{c}R^{d},
   (5) NHC(O)OR^{b},
   (6) NHC(O)NRCR^{d},
   (7) CH₂OR^{a},
   (8) CH₂OCO₂R^{b},
   (9) CH₂OC(O)NR^{c}R^{d},
   (10) C(O)NR^{c}NR^{c}R^{d}, or
   (11) C(O)NR^{c}SO₂R^{b}; [symbol] represents a single or a double bond;
R^{a}
   (1) hydrogen,
   (2) optionally substituted alkyl,
   (3) optionally substituted alkenyl,
   (4) optionally substituted alkynyl,
   (5) optionally substituted alkanoyl,
   (6) optionally substituted alkenoyl,
   (7) optionally substituted alkynoyl,
   (8) optionally substituted aroyl,
   (9) optionally substituted aryl,
   (10) optionally substituted cycloalkanoyl,
   (11) optionally substituted cycloalkenoyl,
   (12) optionally substituted alkylsulfonyl,
   (13) optionally substituted cycloalkyl,
   (14) optionally substituted cycloalkenyl,
   where the substituents on the alkyl, alkenyl, alkynyl, alkanoyl, alkenoyl, alkynoyl, aroyl, aryl, cycloalkanoyl, cycloalkenoyl, alkylsulfonyl, cycloalkyl and cycloalkenyl are from 1 to 10 groups independently selected from hydroxy, alkoxy, cycloalkyl, arylalkoxy, NR⁸R^{h}, CO₂R_{b}, CONR^{c}R^{d} and halogen,

are from 1 to 10 groups independently selected from hydroxy, NR⁸R^{h}, CO₂R_{b}, CONR^{c}R^{d} and halogen
(15) perfluoroalkyl,
(16) arylsulfonyl optionally substituted with 1 to 3 groups independently selected from alkyl, perfluoroalkyl, nitro, halogen and cyano,
(17) a 5- or 6-member heterocycle containing 1 to 4 heteroatoms selected from oxygen, sulfur and nitrogen optionally substituted by 1 to 4 groups independently selected from alkyl, alkenyl, perfluoroalkyl, amino, C(O)NR^{c}R^{d}, cyano, CO₂R^{b} and halogen, and which may be saturated or partly unsaturated;
R^{b} is
   (1) H,
   (2) optionally substituted aryl,
   (3) optionally substituted alkyl,
   (4) optionally substituted alkenyl,
   (5) optionally substituted alkynyl,
   (6) optionally substituted cycloalkyl,
   (7) optionally substituted cycloalkenyl, or
   (8) optionally substituted heterocycle containing from 1 to 4 heteroatoms independently selected from oxygen, sulfur and nitrogen;
   where the substituents on the aryl, alkyl, alkenyl, cycloalkyl, cycloalkenyl, heterocycle, or alkynyl are from 1 to 10 groups independently selected from
      (i) hydroxy,
      (ii) alkyl,
      (iii) oxo,
      (iv) SO₂NR^{g}R^{b},
      (v) arylalkoxy,
      (vi) hydroxyalkyl,
      (vii) alkoxy,
      (viii) hydroxyalkoxy,
      (ix) aminoalkoxy,
      (x) cyano,
      (xi) mercapto,
      (xii) alkyl-S(O)ₘ,
      (xiii) cycloalkyl optionally substituted with 1 to 4 groups independently selected from R^{c},
      (xiv) cycloalkenyl,
      (xv) halogen,
      (xvi) alkanoyloxy,
      (xvii) C(O)NR^{g}R^{h},
      (xviii) CO₂Rⁱ,
      (xix) formyl,
      (xx) —NR^{g}R^{h},
      (xxi) 5-to 9-member heterocycle, which may be saturated or partially unsaturated, containing from 1 to 4 heteroatoms independently selected from oxygen, sulfur and nitrogen, and optionally substituted with 1 to 5 groups independently selected from R^{e},
      (xxii) optionally substituted aryl, wherein the aryl substituents are 1,2-methylenedioxy or 1 to 5 groups independently selected from R^{e},
      (xxiii) optionally substituted arylalkoxy,
   wherein the aryl substituents are 1,2-methylenedioxy or 1 to 5 groups independently selected from R^{c}, and
      (xxiv) perfluoroalkyl,
R^{c} and R^{d} are independently selected from R^{b}; or
R^{c} and R^{d} together with the N to which they are attached form a 3- to 10-member ring, containing 0 to 2 additional heteroatoms selected from O, S(O)ₘ, and N, optionally substituted with 1 to 3 groups independently selected from R^{g}, hydroxy, thioxo and oxo;
R^{e} is
   (1) halogen,
   (2) alkyl,
   (3) perfluoroalkyl,
   (4) —S(O)ₘRⁱ,
   (5) cyano,
   (6) nitro,
   (7) R₁₀(CH₂)v−,
   (8) RⁱCO₂(CH₂)v−,
   (9) RⁱOCO(CH₂)v−,
   (10) optionally substituted aryl where the substituents are from 1 to 3 of halogen, alkyl, alkoxy, or hydroxy,
   (11) SO₂NR^{g}R^{h}, or
   (12) amino;
R^{f} is
   (1) alkyl,
   (2) X—C₁-C₄ alkyl, where X is O or S(O)ₘ,
   (3) alkenyl,
   (4) alkynyl,
   (5) perfluoroalkyl,
   (6) NY¹Y², where Y¹ and Y² are independently H or alkyl,
   (7) hydroxy,
   (8) halogen, and
   (9) alkanoylamino;
R^{g} and R^{h} are independently
   (1) hydrogen,
   (2) alkyl, optionally substituted with hydroxy, amino, or CO₂Rⁱ
   (3) aryl optionally substituted with halogen, 1,2-methylenedioxy, alkoxy, alkyl, or perfluoroalkyl,
   (4) arylalkyl, wherein the aryl is optionally substituted with perfluoroalkyl, or 1,2-methylenedioxy;

(5) alkoxycarbonyl,
(6) alkanoyl,
(7) alkanoylalkyl,
(9) aryl alkoxycarbonyl,
(10) aminocarbonyl,
(11) monoalkylaminocarbonyl,
(12) dialkylaminocarbonyl; or
R^{g} and R^{h} together with the N to which they are attached form a 3- to 7-member ring, containing 0 to 2 additional heteroatoms selected from O, S(O)ₘ, and N, optionally substituted with 1 to 3 groups independently selected from R^{e} and oxo;
Rⁱ is
   (1) hydrogen,
   (2) perfluoroalkyl,
   (3) alkyl,
   (4) optionally substituted aryl or arylalkyl, where the aryl substituents are from 1 to 3 groups independently selected from halogen, alkyl, alkoxy, and hydroxy;
m is 0 to 2; and
v is 0 to 3;or
   a pharmaceutically acceptable salt thereof.

In a preferred embodiment the present invention provides compounds of Formula I′ wherein
R₁ is
   (1) hydrogen,
   (2) optionally substituted alkyl,
   (3) optionally substituted alkenyl,
   (4) optionally substituted alkynyl,
   (5) optionally substituted cycloalkyl,
   (6) optionally substituted cycloalkenyl where the substituents on the alkyl, alkenyl, alkynyl, cycloalkyl and cycloalkenyl are 1 to 3 groups independently selected from.
      (i) alkyl,
      (ii) X—C₁-C₆ alkyl, where X is O or S(O)ₘ,
      (iii) cycloalkyl,
      (iv) hydroxy,
      (v) halogen,
      (vi) cyano,
      (vii) carboxy, and
      (viii) NY¹Y², where Y¹ and Y² are independently H or alkyl,
   (7) aryl or arylalkyl wherein said aryl is optionally substituted with 1 to 3 groups independently selected from R^{f},
   (8) perfluoroalkyl,
   (9) a 5- or 6-member heterocycle containing from 1 to 4 heteroatoms independently selected from oxygen, sulfur and nitrogen atoms optionally substituted by 1 to 3 groups independently selected from hydroxy, oxo, alkyl and halogen, and which may be saturated or partly unsaturated,
R₈ is
   (1) H,
   (2) OH, or
   (3) NH₂;
R₉ is
   (1) H or
   (2) OH;
R₁₀ is
   (1) C(O)OR^{b},
   (2) C(O)N(OR^{b})R^{c},
   (3) C(O)NR^{c}R^{d},
   (4) NHC(O)OR^{b},
   (5) NHC(O)NR^{c}R^{d},
   (6) CH₂OR^{a},
   (7) CH₂OCO₂R^{b},
   (8) CH₂OC(O)NR^{c}R^{d},
   (9) C(O)NR^{c}NR^{c}R^{d}, or
   (10) C(O)NR^{c}SO₂R^{b};
R⁸ is
   (1) hydrogen,
   (2) optionally alkyl,
   (3) optionally substituted alkenyl,
   (4) optionally substituted alkynyl,
   (5) optionally substituted alkanoyl,
   (6) optionally substituted alkenoyl,
   (7) optionally substituted alkynoyl,
   (8) optionally substituted aroyl,
   (9) optionally substituted aryl,
   (10) optionally substituted cycloalkanoyl,
   (11) optionally substituted cycloalkenoyl,
   (12) optionally substituted alkylsulfonyl
   (13) optionally substituted cycloalkyl
   (14) optionally substituted cycloalkenyl where the substituents on the alkyl, alkenyl, alkynyl, alkanoyl, alkenoyl, alkynoyl, aroyl, aryl, cycloalkanoyl, cycloalkenoyl, alkylsulfonyl, cycloalkyl and cycloalkenyl are from 1 to 10 groups independently selected from hydroxy, alkoxy, cycloalkyl, aryl alkoxy, NR^{g}R^{h}, CO₂R^{b}, CONR^{c}R^{d} and halogen,
   (15) perfluoroalkyl,
   (16) arylsulfonyl optionally substituted with 1 to 3 groups independently selected from alkyl, perfluoroalkyl, halogen and cyano,
   (17) a 5- or 6-member heterocycle containing 1 to 4 heteroatoms selected from oxygen, sulfur and nitrogen optionally substituted by 1 to 4 groups independently selected from alkyl, alkenyl, perfluoroalkyl, amino, C(O)NR^{c}R^{d} cyano, CO₂R^{b} and halogen, and which may be saturated or partly unsaturated;
R^{b} is
   (1) H,
   (2) optionally substituted aryl,
   (3) optionally substituted alkyl,
   (4) optionally substituted alkenyl,
   (5) optionally substituted alkynyl,
   (6) optionally substituted cycloalkyl,
   (7) optionally substituted cycloalkenyl, or
   (8) optionally substituted 5- to 10-member heterocycle containing from 1 to 4 heteroatoms independently selected from oxygen, sulfur and nitrogen; where the substituents on the aryl, alkyl, alkenyl, cycloalkyl, cycloalkenyl, heterocycle, or alkynyl are from 1 to 10 groups independently selected from
      (i) hydroxy,
      (ii) C₁-C₃ alkyl,
      (iii) oxo,
      (iv) SO₂NR^{g}R^{h},
      (v) aryl alkoxy,
      (vi) hydroxy alkyl,
      (vii) alkoxy,
      (viii) hydroxyalkoxy,
      (ix) aminoalkoxy,
      (x) cyano,
      (xi) perfluoroalkyl,
      (xii) alkyl—S(O)ₘ,
      (xiii) cycloalkyl optionally substituted with 1 to 4 groups independently selected from R^{e},
      (xiv) cycloalkenyl,
      (xv) halogen,
      (xvi) alkanoyloxy,
      (xvii) C(O)NR^{g}R^{h},
      (xviii) CO₂Rⁱ,
      (xix) optionally substituted arylalkoxy,
      wherein the aryl substituents are 1,2-methylenedioxy or 1 to 5 groups independently selected from R^{c},
      (xx) —NR^{g}R^{h},
      (xxi) 5 to 6-member heterocycle, which may be saturated or partially unsaturated, containing from 1 to 4 heteroatoms independently selected from oxygen, sulfur and nitrogen, and optionally substituted with 1 to 5 groups independently selected from R^{c}, and
      (xxii) optionally substituted aryl, wherein the aryl substituents are 1,2-methylenedioxy or 1 to 5 groups independently selected from R^{e};
R^{c} is
   (1) halogen,
   (2) alkyl,
   (3) perfluoroalkyl,
   (4) —S(O)ₘRⁱ,
   (5) cyano,
   (6) amino,
   (7) RⁱO(CH₂)ᵥ−,
   (8) RⁱCO₂(CH₂)ᵥ−,
   (9) RⁱOCO(CH₂)ᵥ−,
   (10) optionally substituted aryl where the substituents are from 1 to 3 of halogen, alkyl, alkoxy, or hydroxy, or
   (11) SO₂NR^{g}R^{h};
R^{f} is
   (1) methyl,
   (2) X—C1-C2 alkyl, where X is O or S(O)ₘ,
   (3) halogen,
   (4) acetylamino,
   (5) trifluoromethyl,
   (6) NY¹Y², where Y¹ and Y² are independently H or methyl, and
   (7) hydroxy;
R^{g} and R^{h} are independently
   (1) hydrogen,
   (2) alkyl optionally substituted with hydroxy, amino, or CO₂Rⁱ
   (3) aryl optionally substituted with halogen, 1,2-methylenedioxy, alkoxy, alkyl or perfluoroalkyl,
   (4) arylalkyl, wherein the aryl is optionally substituted with perfluorolkyl or 1,2-methylenedioxy;
   (5) alkoxycarbonyl,
   (6) alkanoyl,
   (7) alkanoyl alkyl,
   (9) arylalkoxycarbonyl,
   (10) aminocarbonyl,
   (11) monoalkylaminocarbonyl
   (12) dialkylaminocarbonyl; or
R^{g} and R^{h} together with the N to which they are attached form a 5- to 6-member ring containing 0 to 2 additional heteroatoms selected from O, S(O)ₘ, and N, optionally substituted with 1 to 3 groups independently selected from R^{e} and oxo;
Rⁱ is
   (1) hydrogen,
   (2) perfluoroalkyl,
   (3) alkyl,
   (4) optionally substituted arylalkyl, where the aryl substituents are from 1 to 3 groups independently selected from halogen, alkyl, alkoxy, and hydroxy; all other variables are as defined under Formula I.

In another preferred embodiment, the present invention provides compounds of Formula I′ wherein
Rⁱ is
   (1) hydrogen,
   (2) optionally substituted alkyl,
   (3) optionally substituted alkenyl,
   (4) optionally substituted alkynyl,
   where the substituents on the alkyl, alkenyl, and alkynyl are 1 to 3 groups independently selected from
      (i) methyl,
      (ii) X-methyl, where X is O or S(O)ₘ and
      (iii) halogen,
   (5) aryl or arylalkyl wherein said aryl is optionally substituted with 1 to 3 groups independently selected from R^{f}.
   (6) trifluoromethyl
R₈ is
   (1) H,
   (2) OH, or
   (3) NH₂
R₉is
   (1) H, or
   (2) OH;
R₁₀ is
   (1) C(O)OR^{b},
   (2) C(O)N(OR^{b})R^{c},
   (3) C(O)NR^{c}R^{d},
   (4) NHC(O)OR^{b},
   (5) NHC(O)NR^{c}R^{d},
   (6) CH₂OR^{a},
   (7) CH₂OCO₂R^{b},
   (8) CH₂OC(O)NR^{c}R^{d},
   (9) C(O)NR^{c}NR^{c}R^{d}, or
   (10) C(O)NR^{c}SO₂R^{b};
R^{a} is
   (1) hydrogen,
   (2) optionally substituted alkyl,
   (3) optionally substituted alkenyl,
   (4) optionally substituted alkynyl,
   (5) optionally substituted alkanoyl,
   (6) optionally substituted aroyl,
   (7) optionally substituted cycloalkanoyl,
   (8) optionally substituted cycloalkenoyl,
   (9) optionally substituted alkylsulfonyl
   where the substituents on the alkyl, alkenyl, alkynyl, alkanoyl, aroyl, cycloalkanoyl, cycloalkenoyl, and alkylsulfonyl, are from 1 to 5 groups independently selected from hydroxy, alkoxy, aryl alkoxy, NR^{g}R^{h}, CO₂R^{b}, CONR^{c}R^{d} and halogen,
   (10) trifluoromethyl,
   (11) arylsulfonyl optionally substituted with 1 to 3 groups independently selected from methyl, trifluoromethyl and halogen,
   (12) a 5- or 6-member heterocycle containing 1 to 4 heteroatoms selected from oxygen, sulfur and nitrogen optionally substituted by 1 to 4 groups independently selected from methyl, trifluoromethyl, C(O)NR^{c}R^{d}, CO₂R^{b} and halogen, and which may be saturated or partly unsaturated;
R_{b} is
   (1) H,
   (2) optionally substituted aryl,
   (3) optionally substituted alkyl,
   (4) optionally substituted alkenyl,
   (5) optionally substituted alkynyl,
   (6) optionally substituted cycloalkyl,
   (7) optionally substituted cycloalkenyl, or
   (8) optionally substituted 5- to 6-member heterocycle containing from 1 to 4 heteroatoms independently selected from oxygen, sulfur and nitrogen; where the substituents on the aryl, alkyl, alkenyl, cycloalkyl, cycloalkenyl, heterocycle, or alkynyl are from 1 to 10 groups independently selected from
      (i) hydroxy,
      (ii) alkyl,
      (iii) oxo,
      (iv) SO₂NR^{g}R^{h},
      (v) arylalkoxy,
      (vi) hydroxyalkyl,
      (vii) alkoxy,
      (viii) hydroxy alkoxy,
      (ix) amino alkoxy,
      (x) cyano,
      (xi) alkyl-S(O)ₘ,
      (xii) cycloalkyl optionally substituted with 1 to 4 groups independently selected from R^{e},
      (xiii) cycloalkenyl,
      (xiv) halogen,
      (xv) alkanoyloxy,
      (xvi) C(O)NR^{g}R^{h},
      (xvii) CO₂Rⁱ,
      (xvii) —NR^{g}R^{h},
      (xix) 5 to 6-member heterocycle, which may be saturated or partially unsaturated, containing from 1 to 4 heteroatoms independently selected from oxygen, sulfur and nitrogen, and optionally substituted with 1 to 5 groups independently selected from R^{e},
      (xx) optionally substituted aryl, wherein the aryl substituents are 1,2-methylenedioxy or 1 to 5 groups independently selected from R^{e},
      (xxi) optionally substituted aryl alkoxy, wherein the aryl substituents are 1,2-methylenedioxy or 1 to 5 groups independently selected from R^{e}, and
      (xxii) perfluoroalkyl;
R^{e} is
   (1) halogen,
   (2) alkyl,
   (3) perfludroalkyl,
   (4) —S(O)ₘRⁱ,
   (5) cyano,
   (6) RⁱO(CH₂)ᵥ−,
   (7) RⁱCO2(CH₂)ᵥ−,
   (8) R₁₀CO(CH₂)ᵥ−,
   (9) optionally substituted aryl where the substituents are from 1 to 3 of halogen, alkyl, alkoxy, or hydroxy,
   (10) SO₂NR^{g}R^{h}, or
   (11) amino;
R^{f} is
   (1) methyl,
   (2) X—C₁-C₂ alkyl, where X is O or S(O)ₘ,
   (3) trifluoromethyl,
   (4) NY¹Y², where Y¹ and Y² are independently H or methyl,
   (5) hydroxy,
   (6) halogen, and
   (7) acetylamino,
R^{g} and R^{h} are independently
   (1) hydrogen,
   (2) alkyl optionally substituted with hydroxy, amino, or CO₂Rⁱ
   (3) aryl optionally substituted with halogen, 1,2-methylenedioxy, alkoxy, alkyl or perfluoroalkyl,
   (4) arylalkyl, wherein the aryl is optionally substituted with perfluorolkyl or 1,2-methylenedioxy;
   (5) alkoxycarbonyl,
   (6) alkanoyl,
   (7) alkanoylalkyl,
   (9) arylalkoxycarbonyl,
   (10) aminocarbonyl,
   (11) monoalkylaminocarbonyl
   (12) dialkylaminocarbonyl; or
R^{g} and R^{h} together with the N to which they are attached form a 5-to 6-membered ring containing 0 to 2 additional heteroatoms selected from O, S(O)ₘ, and N, optionally substituted with 1 to 3 groups independently selected from R^{e} and oxo;
Rⁱ is
   (1) hydrogen,
   (2) perfluoroalkyl,
   (3) alkyl,
   (4)optionally substituted aryl or arylalkyl, where the aryl substituents are from 1 to 3 groups independently selected from halogen, alkyl, alkoxy, and hydroxy; and all other variables are as defined under Formula I′.

Most especially preferred are formulations, wherein the composition comprises nodulisporic acid derivatives which are nodulisporamides, which are compounds of the formula 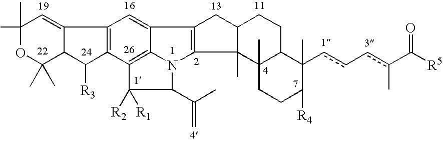
R₁ is
   (1) hydrogen,
   (2) optionally substituted C₁-C₁₀ alkyl,
   (3) optionally substituted C₂-C₁₀ alkenyl,
   (4) optionally substituted C₂-C₁₀ alkynyl,
   (5) optionally substituted C₃-C₈ cycloalkyl,
   (6) optionally substituted C₅-C₈ cycloalkenyl
   where the substituents on the alkyl, alkenyl, alkynyl, cycloalkyl and cycloalkenyl are 1 to 3 groups independently selected from C₁-C₅ alkyl, C₁-C₁₀ alkoxy, C₁-C₁₀ alkylthio, C₁-C₁₀ alkylsulfonyl, C₃-C₈ cycloalkyl, hydroxy, halogen, cyano, carboxy, amino, C₁-C₁₀ monoalkylamino, C₁-C₁₀ dialkylamino, C₁-C₁₀ alkanoyl amino and benzoyl amino wherein said benzoyl is optionally substituted with 1 to 3 groups independently selected from C₁-C₄ alkyl, C₁-C₄ alkoxy, C₁-C₄ alkylthio, C₂-C₄ alkenyl, C₂-C₄ alkynyl, C₁-C₃-perfluoroalkyl, amino, hydroxy, halogen, C₁-C₅ monoalkylamino, C₁-C₅ dialkylamino and C₁-C₅ alkanoyl amino,
   (7) phenyl C₀-C₅ alkyl wherein said phenyl is optionally substituted with 1 to 3 groups independently selected from C₁-C₄ alkyl, C₁-C₄ alkoxy, C₁-C₄ alkylthio, C₂-C₄ alkenyl, C₂-C₄ alkynyl, C₁-C₃₋ perfluoroalkyl, amino, hydroxy, carboxy, halogen, C₁-C₅ monoalkylamino, C₁-C₅ dialkylamino and C₁-C₅ alkanoyl amino,
   (8) C₁-C₅ perfluoroalkyl,
   (9) a 5- or 6-member ring selected from morpholino, pyridyl and piperazino, optionally substituted by 1 to 3 groups independently selected from hydroxy, oxo, C₁-C₁₀ alkyl and halogen,
R², R³, and R⁴ are independently OR^{a}, OCO₂R^{b}, OC(O)NR^{c}R^{d}; or
R¹ and R² together represent ═O, ═NOR^{a} or ═N—NR^{c}R^{d};
R⁵ is NR^{c}R^{d},
R^{a} is
   (1) hydrogen,
   (2) optionally substituted C₁-C₁₀ alkyl,
   (3) optionally substituted C₃-C₁₀ alkenyl,
   (4) optionally substituted C₃-C₁₀ alkynyl,
   (5) optionally substituted C₁-C₁₀ alkanoyl,
   (6) optionally substituted C₁-C₁₀ alkenoyl,
   (7) optionally substituted C₁-C₁₀ alkynoyl,
   (8) optionally substituted benzoyl,
   (9) optionally substituted phenyl,
   (10) optionally substituted C₁-C₇ cycloalkanoyl,
   (11) optionally substituted C₄-C₇ cycloalkenoyl,
   (12) optionally substituted C₁-C₁₀ alkylsulfonyl
   (13) optionally substituted C₃-C₈ cycloalkyl
   (14) optionally substituted C₅-C₈ cycloalkenyl
   where the substituents on the alkyl, alkenyl, alkynyl, alkanoyl, alkenoyl, alkynoyl, benzoyl, phenyl, cycloalkanoyl, cycloalkenoyl, alkylsulfonyl, cycloalkyl and cycloalkenyl are from 1 to 5 groups independently selected from hydroxy, C₁-C₆ alkoxy, C₃-C₇ cycloalkyl, aryl C₁-C₃ alkoxy, NR^{g} R^{h}, CO₂R^{b}, CONR^{c} R^{d} and halogen,
   (15) C₁-C₅ perfluoroalkyl,
   (16) phenylsulfonyl optionally substituted with 1 to 3 groups independently selected from C₁-C₅ alkyl, C₁-C₅ perfluoroalkyl, nitro, halogen or cyano,
   (17) a 5- or 6-member ring selected from piperidino, morpholino, pyridyl and piperazino optionally substituted by 1 to 4 groups independently selected from C₁-C₅ alkyl, C₁-C₅ alkenyl, C₁-C₅ perfluoroalkyl, amino, C(O)R^{c} R^{d}, cyano, CO₂R^{b} or halogen;
R^{b} is
   (1) H,
   (2) optionally substituted phenyl,
   (3) optionally substituted C₁-C₁₀ alkyl,
   (4) optionally substituted C₃-C₁₀ alkenyl, or
   (5) optionally substituted C₃-C₁₀ alkynyl,
   where the substituents on the phenyl, alkyl, alkenyl or alkynyl are from 1 to 5 groups independently selected from hydroxy, C₁-C₆ alkoxy, C₃-C₇ cycloalkyl, halogen, C₁-C₅ alkanoyloxy, C(O)NR^{c}R^{d}, CO₂R^{b}, formyl, —NR^{g} R^{h}, optionally substituted phenyl, and optionally substituted phenyl C₁-C₃ alkoxy, wherein the phenyl substituents are 1 to 3 groups independently selected from R^{e};
R^{c} and R^{d} are independently R^{b}; or
R^{c} and R^{d} together with the N to which they are attached form a piperidino, morpholino or piperazino optionally substituted with 1 to 3 groups independently selected from R^{g} and oxo;
R^{e} is
   (1) halogen,
   (2) C₁-C₇ alkyl,
   (3) C₁-C₃ perfluoroalkyl,
   (4) —S(O)ₘRⁱ,
   (5) cyano,
   (6) nitro,
   (7) R^{j}O(CH₂)ᵥ−,
   (8) R^{j}CO₂ (CH₂)ᵥ −,
   (9) R^{j} OCO(CH₂)ᵥ⁻,
   (10) optionally substituted phenyl where the substituents are from 1 to 3 halogen, C₁ C₆ alkyl, C₁-C₆ alkoxy, or hydroxy;
v is 0 to 3;
R^{g} and R^{h} are independently
   (1) hydrogen,
   (2) C₁-C₆alkyl,
   (3) aryl,
   (4) aryl C₁-C₆ alkyl;
   (5) C₁-C₅ alkoxycarbonyl,
   (6) C₁-C₅ alkylcarbonyl, or
   (7) C₁-C₅ alkanoyl C₁-C₅ alkyl; or
R^{g} and R^{h} together with the N to which they are attached form a piperidino, morpholino or piperazino optionally substituted with 1 to 3 groups independently selected from R^{g} and oxo;
Rⁱ and R^{j} are independently,
   (1) hydrogen,
   (2) C₁-C₃ perfluoroalkyl,
   (3) optionally substituted C₁-C₆ alkyl, where the substituents are aryl or substituted phenyl;
   (4) phenyl or substituted phenyl where the substituents are from 1 to 3 groups independently selected from halogen, Cₗ-C₆ alkyl, C₁-C₆ alkoxy, or hydroxy;
m is 0 to 2; or a pharmaceutically acceptable salt thereof

Most especially preferred are compounds of the formula 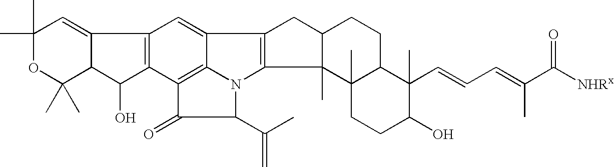
wherein R^{x} is selected from the group consisting of:
H, CH₃, CH₂CH₃, C(CH₃)₃, CH₂CH₂CH₃, CH₂CH₂OH, CH(CO₂CH₃)CH₂OH, CH₂CO₂CH₃, CH₂CH(OCH₂CH₃)₂, CH₂CH₂OCH₂CH₂OH, CH(CH₃)(CH₂)₃C(CH₃)₂OH, (CH₂)₃OH, (CH₂)₄OH, (CH₂)SOH, CH(CH₂OH)CH₂CH₃, NHC(CH₃)₃, CH₂CN, (CH₂)₆OH, CH₂CH(OH)CH₃, CH(CH₂OH)CH₂CH₂CH₃, CH₂CH₂SCH₃, CH₂CH₂SCH₂CH₃, CH₂CONH, CH(CH₃)(CH₂OH)₂, CH₂CH₂NHCH₂CH₂OH, CH(CH₂OH)(CH₂)₃CH₃, CH(CH₂OCH₃)CH₃, (CH₂)₂SH, (CH₂)₄NH₂, CH₂CH₂SO₂CH₃, CH₂CH₂S(O)CH₃, CH(CH(CH₃)₂)CH₂OH, (CH₂)₃NH₂, (CH₂)₃N(CH₂CH₃)₂, (CH₂)₃N(CH₃)₂, OCH₂CH₃, CH₂CH(OH)CH₂OH, OCH₃, CH₂CH₂OCH₃, CH₂CH₂NHC(O)CH₃, C(CH₃)₂CH₂OH, c-C₃H₅, c-C₆H₁₁, (CH₂)₃OCH₂CH₃, CH₂CH≡CH₂, C(CH₂CH₃)(CH₂OH)₂, CH₂C≡CH, CH₂CO₂CH₂CH₃, CH₂CH₂F, (CH₂)₃OCH₂)₁₁ CH₃, CH₂CH₂N(CH₃)₂, CH₂CH₂OCH₂CH₂NH₂, CH₂CF₃, NHCH₂CO₂CH₂CH₃, CH(CH₃)CO₂CH₃, C(CH₃)₂CH₂C(O)CH₃, CH(CO₂CH₂CH₃)₂, CH₂CH₃, CH(CH₂CH₂CH₃)CO₂CH₃, CH₂CH₂CH₂OCH₃, C(CH₃)₂C≡CH, (CH₂)₄CH₃, CH(CH₂CH₂CH₃)₂, (CH₂)₅CH₃,CH₂CH₂CO₂H, CH(CH(CH₃)₂)CO₂CH₃, OCH₂CO₂H, CH(CH(CH₃)₂)CH₂OH, CH(CH(CH₃)₂)CH₂OH, CH(CH₃)CH₂OH, CH(CH₃)CH₂OH, CH(CH₃)₂, C(CH₃)₃, (CH₂)CH(CH₃)₂, CH(CH₃)CH₂CH₃, CH₂CH(CH₃)OH, (CH₂)₃CH₃, (CH₂)₂OCH₂CH₃, 1-adamantyl, (CH₂)₈CH₃, CH(CH₃)CH(CH₃)₂, (CH₂)₃NHCH₃, (CH₂)₂N(CH₂CH₃)₂,

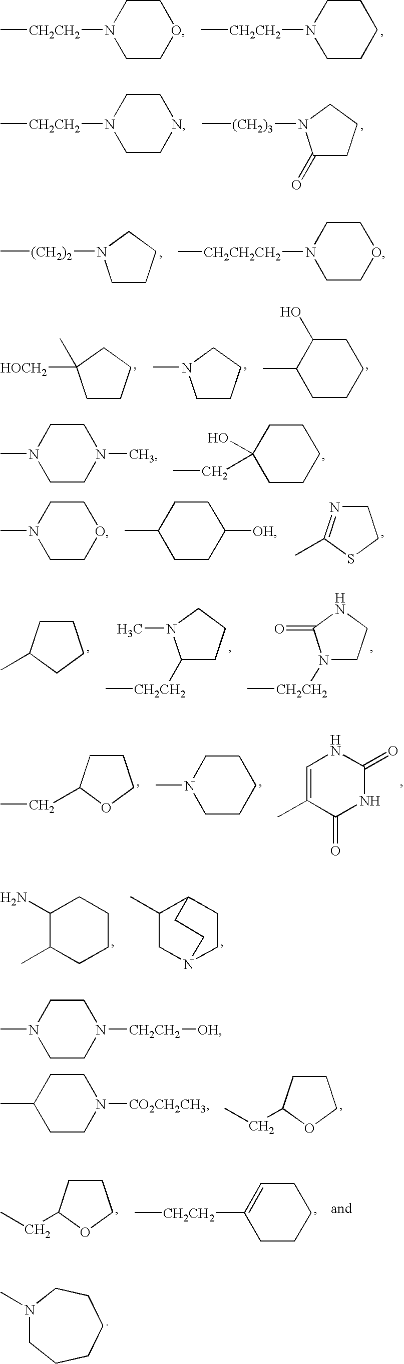

An especially preferred nodulisporamide derivative is one wherein R^{X} is with t-butyl (or “nodulisporamide”).

“Alkyl” as well as other groups having the prefix “alk”, such as alkoxy, alkanoyl, alkenyl, alkynyl and the like, means carbon chains which may be linear or branched or combinations thereof. Examples of a ikyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as benzofused carbocycles. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofised cycloalkenes. Examples of cycloalkenyl include cyclohexenyl, indenyl, and the like.

The term “halogen” is intended to include the halogen atoms fluorine, chlorine, bromine and iodine.

The term “heterocycle”, unless otherwise specified, means mono- or bicyclic compounds that are saturated or partly unsaturated, as well as benzo- or heteroaromatic ring fused saturated heterocycles or partly unsaturated heterocycles, and containing from 1 to 4 heteroatoms independently selected from oxygen, sulfur and nitrogen. Examples of saturated heterocycles include morpholine, thiomorpholine, piperidine, piperazine, tetrahydropyran, tetrahydrofuran, dioxane, tetrahydrothiophene, oxazolidine, pyrrolidine; examples of partly unsaturated heterocycles include dihydropyran, dihydropyridazine, dihydrofuran, dihydrooxazole, dihydropyrazole, dihydropyridine, dihydropyridazine and the like. Examples of benzo- or heteroaromatic ring fused heterocycle include 2,3-dihydrobenzofuranyl, benzopyranyl, tetrahydroquinoline, tetrahydroisoquinoline, benzomorpholinyl, 1,4-benzodioxanyl, 2,3-dihydrofuro(2,3-b)pyridyl and the like.

The term “aryl” is intended to include mono- and bicyclic aromatic and heteroaromatic rings containing from 0 to 5 heteroatoms independently selected from nitrogen, oxygen and sulfur. The term “aryl” is also meant to include benzofused cycloalkyl, benzofused cycloalkenyl, and benzofused heterocyclic groups. Examples of “aryl” groups include phenyl, pyrrolyl, isoxazolyl, pyrazinyl, pyridinyl, oxazolyl, thiazolyl, imidazolyl, triazolyl, tetrazolyl, furanyl, triazinyl, thienyl, pyrimidinyl, pyridazinyl, pyrazinyl, naphthyl, benzoxazolyl, benzothiazolyl, benzimidazolyl, benzofuranyl, furo(2,3-B)pyridyl, 2,3-dihydrofuro(2,3-b)pyridyl, benzoxazinyl, benzothiophenyl, quinolinyl, indolyl, 2,3-dihydrobenzofuranyl, benzopyranyl, 1,4-benzodioxanyl, indanyl, indenyl, fluorenyl, 1,2,3,4-tetrahydronaphthalene and the like.

Aroyl means arylcarbonyl in which aryl is as defined above.

Examples of NR^{c}R^{d} or NR^{g}R^{h} forming a 3-to 10-membered ring containing 0 to 2 additional heteroatoms selected from O, S(O)ₘ and N are aziridine, azetidine, pyrrolidine, piperidine, thiomorpholine, morpholine, piperazine, octahydroindole, tetrahydroisoquinoline and the like.

The term “optionally substituted” is intended to include both substituted and unsubstituted; thus, for example, optionally substituted aryl could represent a pentafluorophenyl or a phenyl ring.

Certain of the above defined terns may occur more than once in the above formula and upon such occurrence each term shall be defined independently of the other; thus, for example, OR^{a} at C4 may represent OH.

Compounds of formula (I′) are available commercially or can be prepared according to one or other of the processes or any other process coming within the competence of a person skilled in the art who is an expert in chemical synthesis. For the chemical preparation of the products of the invention, a person skilled in the art is regarded as having at his disposal, inter alia, the entire contents of “Chemical Abstracts” and of the documents, which are cited therein. Semi-synthetic processes are described, for example, in U.S. Pat. No. 6,399,786 or WO 96/29073, both of which are incorporated by reference. The terms “nodulisporic acid or nodulisporic acid derivative” also include the pharmaceutically or veterinary acceptable acid or base salts, where applicable, of these compounds.

### BRIEF DESCRIPTION OF THE DRAWINGS

FIG. 1 is a chromatogram of the degradation of the thioamide derivative of fipronil to fipronil under UV light. The degradation reaction is depicted below:

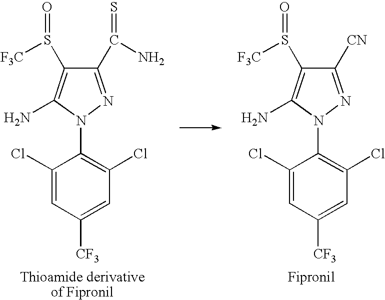

FIG. 2 is a graphical representation of the data presented in Example 3.

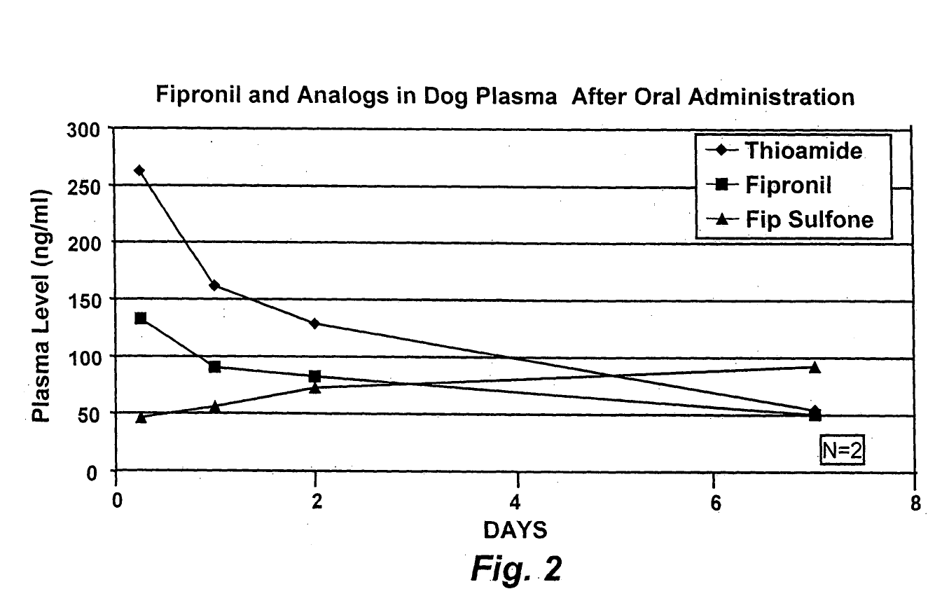

### EXAMPLES

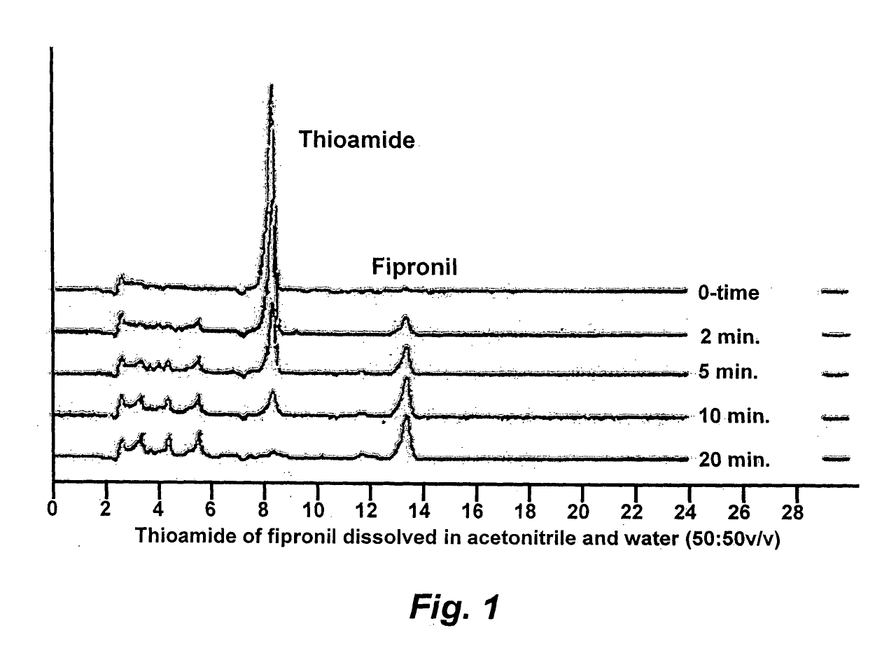

The present invention will now be described in greater detail with the aid of non-limiting embodiment examples which demonstrate the activity of the solutions according to the present invention, with reference to FIG. 1 as describe above.

### Example 1

In Vitro Degradation Studies

A solution containing thioamide derivative of fipronil (“TD-fipronil”) in a solution of CH₃CN/H₂O (50:50 v/v) and the solution was subjected to the following degradation agents: an acid (0.5 N HCl); a base (0.5 N NaOH); fluorescent light; heat (65° C.); an oxidizer (5% H₂O₂); and UV light. The identity of fipronil formed was confirmed via HPLC with UV detection and HPLC with diode array detection. The results are presented below:

| Agent | Comments |
|---|---|
| Acid (0.5 N HCl) | TD-fipronil stable in acid with only 2.7% |
| | being degraded in 4 days; negligible amounts |
| | of fipronil formed. |
| Base (0.5 N NaOH) | 95% of the TD-fipronil degraded in 1 hour, but |
| | no fipronil was formed |
| Fluorescent light | 23% of TD-fipronil degraded to fipronil in 4 |
| | days. |
| Heat (65° C.) | 20% of TD-fipronil degraded to fipronil in 4 |
| | days. |
| Oxidation (5% H₂O₂) | 100% of the TD-fipronil degraded in 1.5 hours |
| | but no fipronil was formed; fipronil sulfone |
| | was formed. |
| UV light | TD-fipronil was very sensitive to light. 24% |
| | of the TD-fipronil degraded in 1 hour |

### Example 2

In Vivo Degradation Study—Topical Administration

A topical spot-on solution of the thioamide solution containing 100 mg/ml of thioamide and diethylene glycol monoethyl ether was applied to the fur of a dog at the dose rate of 0.1 ml/kg of body weight. The concentration of fipronil in the fur was measured on day 2 and day 7.

| | Thioamide of fipronil | Fipronil | Fipronil sulfone |
|---|---|---|---|
| | (μg/g) | (μg/g) | (μg/g) |
| Day 2 | 362.8 | 148.5 | 9.3 |
| Day 7 | 59.3 | 27.8 | 2.7 |

The concentration of fipronil was sufficient for efficacy against fleas and ticks.

### Example 3

In Vivo Degradation Study—Oral Administration

An oral formulation (solution) comprising 100 mg/ml of thioamide derivatives of fipronil dissolved in 60:40 propylene glycol: glycerol formal was orally administered to dogs at a dosage rate of 10 mg/kg and the concentration of fipronil in blood plasma measured at 6 hours, day 1, day 2, and day 7 and is presented below

| | Thioamide of fipronil | Fipronil | Fipronil sulfone |
|---|---|---|---|
| | (μg/g) | (μg/g) | (μg/g) |
| Pre-treatment | 0.0 | 0.0 | 11.3 |
| Day 0.25 (6 hours) | 262.4 | 132.9 | 46.9 |
| Day 1 | 161.9 | 89.5 | 56.1 |
| Day 2 | 128.6 | 82.5 | 72.7 |
| Day 7 | 54.6 | 49.9 | 92.3 |

The data demonstrated that the thioamide derivative of fipronil degrades into fipronil in vivo in amounts that are sufficient for efficacy against fleas and ticks.

### Example 4

A pour-on formulation containing the following ingredients is prepared.

| Ingredient | Function | Amount | |
|---|---|---|---|
| Thioamide derivative of fipronil | active substance | 10 | g |
| polyvidone | crystallization inhibitor | 0.2 | g |
| diethyl glycol monomethyl ether | diluent | qs 100 | ml |

### Example 5

A pour-on formulation containing the following ingredients is prepared.

| Ingredient | Function | Amount | |
|---|---|---|---|
| Thioamide derivative of fipronil | active substance | 10 | g |
| polyoxypropylene 15 stearyl ether | emollient | 2 | g |
| soybean oil | diluent | qs 100 | ml |

### Example 6

A pour-on formulation containing the following ingredients is prepared.

| Ingredient | Function | Amount | |
|---|---|---|---|
| Thioamide derivative of fipronil | active substance | 10 | g |
| polyvidone | crystallization inhibitor | 2 | g |
| miglyol | solvent | qs 100 | ml |

### Example 7

| COMPOSITION | % w/w |
|---|---|
| Active Ingredients: | |
| Thioamide derivative of fipronil | 0.01-20.0 |
| Excipients: | |
| Polyoxyl 40 Hydrogenated Castor Oil | 8.00 |
| Distilled Monoglycerides | 20.80 |
| Formulated Antioxidant:* | 0.5 |
| Butylated Hydroxyanisole (BHA) | 0.10 |
| Propyl Gallate | 0.03 |
| Citric Acid, Anhydrous | 0.02 |
| Propylene Glycol | 0.35 |
| Fine Ground Corn Cobs | QS 100% |

*Sustan 3 ®, a commercially available antioxidant mixture consisting of butylated hydroxyanisol (20% w/w); propyl gallate (6% w/w) and anhydrous citric acid (4% w/w) in a propylene glycol base.

### Example 8

A premix containing the following ingredients is prepared as follows:

| | | |
|---|---|---|
| Thioamide derivative of fipronil | 4.68% | w/w |
| Propylene Glycol | 15% | w/w |
| BHA | 0.10% | w/w |
| Propyl Gallate | 0.03 | w/v |
| Distilled Monoglycerides | 20.8 | w/w |

| | |
|---|---|
| Corn Cobs | QS 100% |

Corn cobs are placed in an oven and heated for ˜3 hours at 75-85° C. Antioxidants, BHA and Propyle Gallate, are dissolved in propylene glycol, following with the addition of the thioamide derivative of fipronil. The mixture is heated to 75-85° C. to dissolve all ingredients and melted monoglycerides are then added at 75-85° C. A hot solution of the thioamide derivative of fipronil is added to heated corncobs and mixed well. The product is cooled.

Alternately, propylene glycol and distilled monoglycerides are heated at 75-85° C. following with the addition of antioxidants (BHA & Propyl Gallate) and the thioamide derivative of fipronil. All ingredients are dissolved while keeping at 75-85° C. The hot mixture is added to the heated corncobs while mixing at 75-85° C. and is then cooled.

### Example 9

The following premix is prepared by using the procedure given in the above Example 8.

| | | |
|---|---|---|
| Thioamide derivative of fipronil | 6.0% | w/w |
| Polyethylene Glycol | 20% | w/w |
| BHA | 0.10% | w/w |
| Propyl Gallate | 0.03 | w/v |
| Distilled Monoglycerides | 20.8 | w/w |

| | |
|---|---|
| Corn Cobs | QS 100% |

### Example 10

The premix is prepared by using the procedure provided above in Example 8.

| | | |
|---|---|---|
| Thioamide derivative of fipronil | 6.0% | w/w |
| Diethylene Glycol Monobutyl Ether | 10% | w/w |
| BHA | 0.10% | w/w |
| Propyl Gallate | 0.03 | w/v |
| Distilled Monoglycerides | 20.8 | w/w |

| | |
|---|---|
| Corn Cobs | QS 100% |

The above description is intended to be illustrative and not limiting. Various changes or modifications in the embodiments described herein may occur to those skilled in the art. These can be made without departing from the scope or spirit of the invention.

What is claimed is:

1. A process for the control or elimination of external parasites from an animal, comprising topically applying or orally administering a parasitically effective amount of a formulation comprising a compound of the formula: 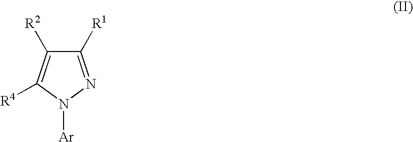
wherein:
R¹ represents H₂N—CS—;
R² represents S(O)ₙR, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of 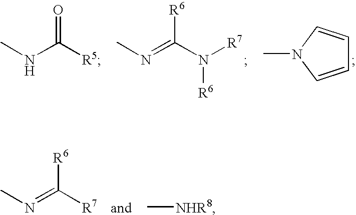
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl,
or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and
n represents a number 0, 1 or 2; and subjecting the formulation to degradation while diffusing therefrom over the animal's body and/or in the sebaceous glands of the animal and thereby obtaining said control or elimination of said external parasites.

2. The process according to claim 1 wherein the degradation is photodegradation.

3. The process according to claim 1 wherein the degradation is thermal degradation.

4. The process according to claim 1, wherein the formulation further comprises a second parasiticide.

5. The process according to claim 4, wherein the second parasiticide is selected from the group consisting of avermectins, milbemycins, IGR compounds, nodulisporic acid, nodulisporic acid derivatives, pyrethroids and formamidines.

6. A process for preparing a compound of the formula 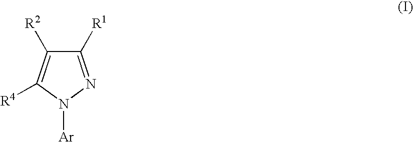
wherein:
R¹ represents C≡N
R² represents S(O)ₙR³, 4,5-dicyanoimideazol-2-yl or haloalkyl;
R³ represents alkyl, haloalkyl, haloalkenyl or halogenalkynyl;
R⁴ represents hydrogen, halogen, alkyl, amino or a compound selected from the group consisting of 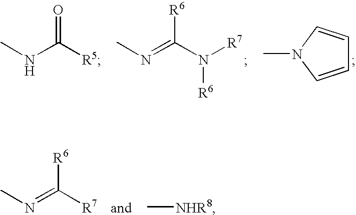
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl,
or a radical NR⁹R⁹R¹⁰, S(O)ₘR¹¹, C(O)R¹¹, C(O)R¹¹, OR¹¹, or —N═C(R¹³)(R¹⁴) wherein R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms; R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents, independently of each other, an integer equal to 0, 1, or 2;
which comprises degrading a compound of the formula: 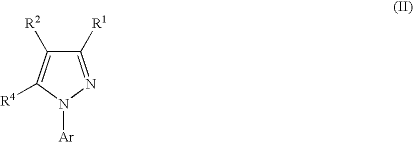
wherein:
R¹ represents H₂N—CS—;
R² represents S(O)ₙR³, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of 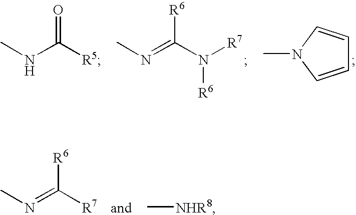
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl, or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and
n represents a number 0, 1 or 2, in the presence of a degradation agent

7. The process according to claim 6, wherein the degradation agent is fluorescent light, UV light or heat.

8. A chewable veterinary formulation, which does not contain animal products, which comprises:
effective amount of at least one compound of the formula; 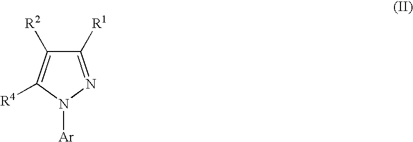
wherein:
R¹ represents H₂N—CS—;
R² represents S(O)ₙR, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of 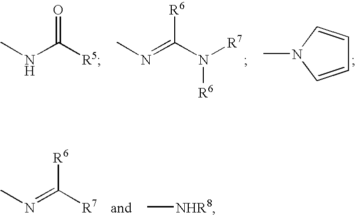
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl, or a radical NR⁹R⁹R¹⁰, S(O)ₘR¹¹, C(O)R¹¹, C(O)R¹¹, OR ¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and n represents a number 0, 1 or 2,
at least one filler;
at least one disintegrant;
at least one non-animal product containing flavor or flavor derived from a non-animal source;
at least one binder;
at least one humectant;
at least one granulating solvent; and
optionally, at least one antioxidant, at least one pH modifier, at least one surfactant, at least one preservative, at least one lubricant or at least one colorant.

9. The chewable veterinary formulation according to claim 8, wherein,
the filler is selected from the group consisting of soy protein, corn cob, and corn gluten meal;
the disintegrant is selected from the group consisting of sodium starch glycolate, crospovidone, croscarmellose sodium, starch, micocrystalline cellulose, alginic acid, veegum, crospovidone, bentonite, and pregelatinized starch;
the binder is selected from the group consisting of polyvinyl pyrrolidone, povidone, starch, pregelatinized starch, gelatin, methylcellulose, hydroxypropyl cellulose, carboxymethyl cellulose sodium, ethylcellulose, sodium alginate, tragacanth, and acacia;
the humectant is selected from the group consisting of propylene glycol, glycerin, and polyethylene glycol 400; and
the granulating solvent is water or an aqueous sorbitol solution.

10. The chewable veterinary formulation according to claim 9, which further comprises an antioxidant and the antioxidant is an alpha tocopherol, ascorbic acid, ascrobyl palmitate, sodium ascorbate, sodium metabisulfate, n-propyl gallate, butylated hydroxy anisole, butylated hydoxy toluene, monothioglycerol or a mixture of any of the foregoing.

11. The chewable veterinary formulation according to claim 10, which further comprises a colorant and the colorant is a dye, an aluminum lake, caramel, colorant based upon iron oxide or a mixture of any of the foregoing.

12. The chewable veterinary formulation according to claim 11, which further comprises a preservative and the preservative is a compound selected from the group consisting of benzalkonium chloride, benzethonium chloride, benzoic acid, benzyl alcohol, bronopol, butylparaben, centrimide, chlorhexidine, chlorobutanol, chlorocresol, cresol, ethylparaben, imidurea, methylparaben, propylparaben, phenol, phenoxyethanol, phenylethyl, alcohol, phenylmercuric acetate, phenylmecuric borate, phenylmercuric nitrate, potassium sorbate, sodium benzoate, sodium propionate, sorbic acid, thimerosal, propyl paraben, myristyl gamma-picolinium chloride, paraben methyl, paraben propyl, quaternary ammonium compounds and a mixture of any of the foregoing.

13. The chewable veterinary formulation according to claim 12, which further comprises a surfactant selected from the group consisting of glyceryl monooleate, polyoxyethylene, sorbitan esters, polyvinyl alcohol, sodium lauryl sulfate and poloxomers.

14. The chewable veterinary formulation according to claim 12, which further comprises a lubricant and the lubricant is selected from the group consisting of corn oil, polyethylene glycol, mineral oil, hydrogenated vegetable oil, peanut oil and castor oil.

15. The chewable veterinary formulation, according to claim 8 which comprises:
an effective amount of a compound of formula (II)
about 20 to about 60% of a filler selected from the group consisting of soy protein, corn cob, or corn gluten meal;
about 1 to about 20% of a disintegrant;
about 0.1 to about 20% of a non-animal product containing flavor or a flavor derived from a non-animal source;

16. The chewable veterinary formulation according to claim 15 wherein the compound of formula II is the thioamide derivative of fipronil.

17. The chewable veterinary formulation according to claim 8, which further comprises a second parasiticide.

18. The chewable veterinary formulation according to claim 17, wherein the second parasiticide is selected from the group consisting of avermectins, milbemycins, IGR compounds, nodulisporic acid, and nodulisporic acid derivatives.

19. A tablet, which does not contain animal products, which comprises
an effective amount of at least one compound of the formula: 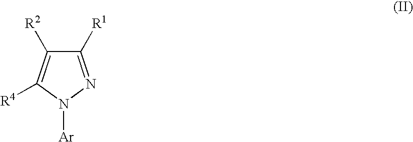
wherein:
R¹ represents H₂N—CS—;
R² represents S(O)ₙR, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of 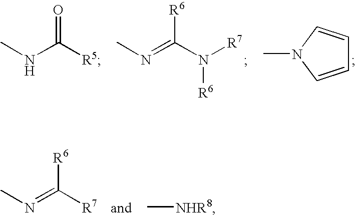
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl, or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O),CF₃ radical; or R⁹ and R¹⁰ may together form a divalent alkenyl radical which may be interrupted by one or two heteroatoms;
R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and n represents a number 0, 1 or 2,
at least one filler;
at least one disintegrant;
at least one non-animal product containing flavor or flavor derived from a non-animal source;
at least one binder;
at least one humectant;
at least one granulating solvent; and
optionally, at least one lubricant, at least one flow aid, at least one antioxidant, at least one pH modifier, at least one surfactant, at least one preservative, at least one lubricant or at least one colorant.

20. The tablet according to claim 19, wherein
the filler is selected from the group consisting of anhydrous lactose, hydrated lactose, spray-dried lactose, crystalline maltose, and a maltodextin;
the flow aid is selected from the group consisting of silicone dioxide, silica gel, talc, starch, calcium stearate, magnesium stearate, and aluminum magnesium stearate; and
the lubricant is selected from the group consisting of magnesium stearate, calcium stearate, stearic acid and waxes.

21. The tablet according to claim 20, wherein
the disintegrant is selected from the group consisting of sodium starch glycolate, crospovidone, croscarmellose sodium, starch, micocrystalline cellulose, alginic acid, veegum, crospovidone, bentonite, and pregelatinized starch; and
the binder is selected from the group consisting of polyvinyl pyrrolidone, povidone, starch, pregelatinized starch, gelatin, methylcellulose, hydroxypropyl cellulose, carboxymethyl cellulose sodium, ethylcellulose, sodium alginate, tragacanth, and acacia.

22. The tablet according to claim 21, which further comprises a colorant and the colorant is a dye, an aluminum lake, caramel, colorant based upon iron oxide or a mixture of any of the foregoing.

23. The tablet according to claim 19, which further comprises a second parasiticide.

24. The tablet according to claim 23, wherein the second parasiticide is selected from the group consisting of avermectins, milbemycins, IGR compounds, formamidines, pyrethroids, nodulisporic acid, and nodulisporic acid derivatives.

25. The tablet according to claim 20 wherein the compound of formula II is the thioamide derivative of fipronil.

26. A premix formulation which comprises
an effective amount of at least one compound of the formula 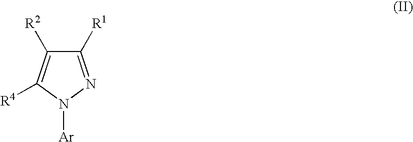
wherein:
R¹ represents H₂N—CS—;
R² represents S(O)ₙR, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of 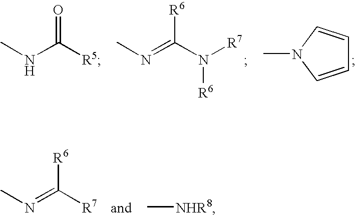
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl, or a radical NR⁹R⁹R¹⁰, S(O)ₘR¹¹, C(O)R¹¹, C(O)R¹¹, OR^{12,} or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and
n represents a number 0, 1 or 2,
a pharmaceutically acceptable excipient comprising:
i) a pharmaceutically acceptable surfactant;
ii) a pharmaceutically acceptable wax;
iii) a pharmaceutically acceptable antioxidant;
iv) a pharmaceutically acceptable carrier vehicle wherein said vehicle is selected from the group consisting of fine corn cobs, corn meal, citrus meal, fermented residues, ground oyster shells, wheat shorts, molasses solubles, bean mill feed, soy grits, crushed limestone and dried grains; and
optionally a pharmaceutically acceptable pH modifier.

27. The premix formulation according to claim 26 which further comprises a second parasiticide.

28. The premix formulation according to claim 27 wherein the second parasiticide is selected from the group consisting of avermectins, milbemycins, IGR compounds, nodulisporic acid and nodulisporic acid derivatives.

29. A process for the control or elimination of external parasites from an animal which comprises adding the premix according to claim 26 to animal feed.

30. A spray which comprises
an effective amount of at least one compound of the formula 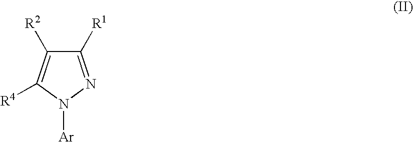
wherein:
R¹ represents H₂N—CS—;
R² represents S(O)ₙR, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of 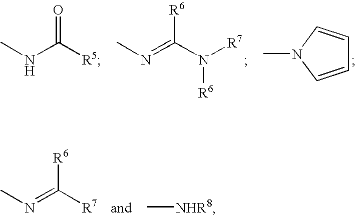
wherein
R⁵ represents alkyl, halogenoalkyl, alkoxyalkyl or in each case unsubstituted or substituted phenyl or pyridyl,
R⁶ represents hydrogen or alkyl,
R⁷ represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl,
or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and
n represents a number 0, 1 or 2,
a vehicle and,
optionally a crystallization inhibitor.

31. The spray according to claim 27, which further comprises a second parasiticide.

32. The spray according to claim 28, wherein the second parasiticide is selected from the group consisting of avermectins, milbemycins, IGR compounds, nodulisporic acid, formamidines, pyrethroids, and nodulisporic acid derivatives.

33. A premix formulation which comprises
an effective amount of at least one compound of the formula 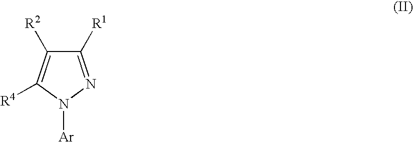
wherein:
R¹ represents H₂N—CS—;
R² represents S(O)ₐR, 4,5-dicyanoimidazol-2-yl or haloalkyl;
R³ represents alkyl, haloalkyl, haloalkenyl or haloalkynyl;
R⁴ represents hydrogen, halogen, alkyl, haloalkyl, amino or a compound selected from the group consisting of 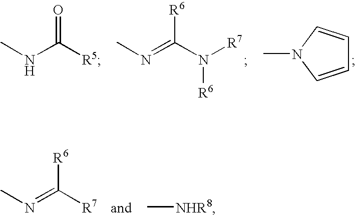
wherein
R⁵ represents alkyl, halogenoalkyl, alkoxyalkyl or in each case unsubstituted or substituted phenyl or pyridyl,
R⁶ represents hydrogen or alkyl,
R⁷represents hydrogen, alkyl or in each case unsubstituted or substituted phenyl or pyridyl,
R⁸ represents alkyl, alkenyl, alkynyl, formyl, alkylcarbonyl, halogenoalkylcarbonyl, or alkoxycarbonyl, or a radical NR⁹R⁹R¹⁰, S(O)ₘR¹¹, C(O)R¹¹, C(O)R¹¹, OR¹², or —N═C(R¹³)(R¹⁴)
wherein
R⁹ and R¹⁰ independently represent a hydrogen atom or an alkyl, haloalkyl, C(O)alkyl, alkyoxycarbonyl or a S(O)ᵣCF₃ radical; or R⁹ and R¹⁰ may together form a divalent alkenyl radical which may be interrupted by one or two heteroatoms;
R¹¹ represent an alkyl or haloalkyl radical;
R¹² represents an alkyl or haloalkyl radical or a hydrogen atom;
R¹³ represents an alkyl radical or a hydrogen atom;
R¹⁴ represents a phenyl or a heteroaryl group optionally substituted with one or more halogen atoms or OH, —O-alkyl, S-alkyl, cyano or alkyl;
m, n q, r represents independently of each other an integer equal to 0, 1, or 2;
Ar represents unsubstituted or substituted phenyl or pyridyl, and
n represents a number 0, 1 or 2,
a pharmaceutically acceptable excipient comprising:
i) a pharmaceutically acceptable wax;
ii) a pharmaceutically acceptable antioxidant;
iii) a pharmaceutically acceptable carrier vehicle wherein said vehicle is selected from the group consisting of fine corn cobs, corn meal, citrus meal, fermented residues, ground oyster shells, wheat shorts, molasses solubles, bean mill feed, soy grits, crushed limestone and dried grains;
an organic solvent and
optionally a pharmaceutically acceptable pH modifier.

34. A process for the control or elimination of external parasites from an animal which comprises spraying the animal with an effective amount of a spray according to claim 27.

35. The process of claim 1 wherein the application is topical.

36. The process of claim 35, wherein the topical application is to a localized region on the back of the animal and the formulation is a spot-on formulation.

37. The process of claim 36, wherein the spot-on formulation comprises from about 0.05 to about 25% weight/volume, relative to the total solution, of a compound of the formula (II).

38. The process of claim 37, wherein the compound of formula (II) is applied in a dose between about 0.1 and about 2 mg/kg animal weight.

39. The process according to claim 35 wherein the degradation is photodegradation.

40. The process according to claim 39 wherein the degradation is thermal degradation.

41. The process according to claim 35 wherein the formulation further comprises a second parasiticide.

42. The process according to claim 41, wherein the second parasiticide is selected from the group consisting of avermectins, milbemycins, IGR compounds, nodulisporic acid, nodulisporic acid derivatives; pyrethroids and formamidines.
